(19) United States
(12) Patent Application Publication
(10) Pub. No.: US 2016/0017318 A1
(43) Pub. Date: Jan. 21, 2016

(54) GENE SIGNATURES TO IDENTIFY MOLECULAR SUBGROUPS IN MEDULLOBLASTOMA TUMORS

(71) Applicant: Children's Hospital Los Angeles, Los Angeles, CA (US)

(72) Inventors: Shahab Asgharzadeh, Los Angeles, CA (US); Ashley Margol, Venice, CA (US); Richard Sposto, Burbank, CA (US)

(73) Assignee: Children's Hospital Los Angeles, Los Angeles, CA (US)

(21) Appl. No.: 14/805,353

(22) Filed: Jul. 21, 2015

Related U.S. Application Data
(60) Provisional application No. 62/027,150, filed on Jul. 21, 2014.

(51) Int. Cl. C12N 15/10

(52) U.S. Cl. CPC C12N 15/1058

(57) ABSTRACT

Described herein are methods for determining the subgroup of medulloblastoma in a subject in need thereof. The subgroups of medulloblastoma include Group 3, Group 4, WNT and SHH. The subjects are children diagnosed with medulloblastoma or children suspected of having medulloblastoma.

### CROSS-REFERENCE TO RELATED APPLICATIONS

This application claims priority under 35 U.S.C. §119(e) to U.S. provisional patent application No. 62/027,150 filed Jul. 21, 2014, currently pending, the contents of which are herein incorporated by reference in its entirety.

### FIELD OF INVENTION

This invention relates to diagnostic assays to identify molecular subgroups of tumors, specifically medulloblastoma.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Medulloblastoma is the most common malignant childhood brain tumor. Approximately 30% of patients remain incurable and current radiation therapy containing treatment protocols cause significant adverse long-term neurocognitive effects and endocrine dysfunction. The role of tumor microenvironment as an enabling characteristic of cancer and development of novel immunotherapeutics invokes the possibility of tumor-associated inflammatory cells as therapeutic targets. Here the inventors report distinct tumor microenvironments of medulloblastoma molecular subgroups and the presence of tumor-associated macrophages (TAMs) in the Sonic Hedgehog subgroup of medulloblastomas. A a 31-gene expression signature inclusive of inflammation-related genes that is clinically applicable and highly accurate in classifying medulloblastoma subgroups, has been developed and described herein. The presence of TAMs is shown using immunohistochemistry and demonstrate their proximity to proliferating cells. The work described herein sheds light on the importance of the tumor microenvironment in childhood brain tumors and inhibition of TAMs, possibly through CSF1R inhibitor, as a potential new therapeutic target in medulloblastomas.

The role of inflammation in promoting tumor growth and regulation of the anti-tumor immune response is an important characteristic of cancer. Tumor-associated macrophages (TAMs) are major contributors to the tumor microenvironment and are present in a variety of human cancers. TAMs are now known to promote cancer via multiple mechanisms including effects on tumor cell growth, survival, invasion, metastasis, angiogenesis, inflammation, and immunoregulation. The presence of TAMs has been described in many adult malignancies including central nervous system tumors. The first evidence of TAMs in childhood tumors and its correlation with tumor stage was recently shown in neuroblastoma, a peripheral nervous system tumor. However little is known about the role of TAMs and the tumor microenvironment in childhood brain tumors.

Medulloblastoma is the most common malignant brain tumor in children. Craniospinal irradiation still remains an essential component of multimodality therapy for many young children putting these patients at risk for developmental neurotoxicity. Medulloblastomas are a molecularly heterogeneous group of tumors that can be classified into at least four distinct subgroups: WNT, SHH, Group 3 and Group 4. Recent data have provided insight into the biology and prognosis of medulloblastomas; however, relatively little is known about the role of the tumor microenvironment with respect to these molecular subgroups. Large-scale genomic and gene expression analyses using a number of different platforms have been shown to accurately identify medulloblastoma subgroups, but implementation in real-time for clinical application remains a challenge.

In this study, the inventors developed a novel and clinically applicable 31-gene TaqMan Low Density Array® (TLDA) signature that allows accurate identification of medulloblastoma subgroups and examined the expression of inflammation-related genes with respect to each subgroup. Evidence of intra-tumoral inflammation and presence of TAMs in the SHH subgroup of medulloblastomas correlating with regions of proliferation was identified. The inventors' findings provide the first report of TAMs in pediatric brain tumors and introduce the possibility of utilizing targeted therapies against TAMs thereby decreasing the reliance on current radiation-based therapies, which are associated with potentially devastating long-term neurocognitive sequelae in young children.

### SUMMARY OF THE INVENTION

The following embodiments and aspects thereof are described and illustrated in conjunction with systems, compositions and methods which are meant to be exemplary and illustrative, not limiting in scope.

Described herein are processes and systems for stratifying subject with medulloblastoma into subgroups so as to optimize treatment.

Described herein is a process, comprising providing a first composition comprising a plurality of isolated nucleic acids probes; contacting the first composition to an RNA sample from a mammalian subject desiring a determination of a subgroup of medulloblastoma, to produce one or more cDNA molecules; providing a second composition comprising one or more isolated nucleic acids probes comprising a sequence capable of hybridizing to one or more nucleic acids selected from the genes set forth in Table 3; contacting the second composition with the one or more cDNA molecules to amplify the one or more cDNA molecules; quantifying the expression level of the one or more genes to determine the subgroup of medulloblastoma to which the sample belongs; and computing the prediction probabilities of the sample belonging the subgroup of medulloblastoma, wherein the subgroup is selected from the group consisting of Group 3, Group 4, SHH and WNT.

In an embodiment, provided herein is a process, comprising: providing a first composition comprising a plurality of isolated nucleic acids probes; contacting the first composition to an RNA sample from a mammalian subject desiring a determination of a subgroup of medulloblastoma, to produce one or more cDNA molecules; providing a second composition comprising one or more isolated nucleic acids probes comprising a sequence capable of hybridizing to one or more nucleic acids selected from the group of genes consisting of βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC, βWIF1 or a combination thereof; contacting the second composition with the one or more cDNA molecules to amplify the one or more cDNA molecules; quantifying the expression level of the one or more genes to determine the subgroup of medulloblastoma to which the sample belongs; and computing the prediction probabilities of the sample belonging to the subgroup of medulloblastoma.

In some embodiments, the subgroup is selected from the group consisting of Group 3, Group 4, SHH and WNT. In some embodiments, amplifying comprises quantitative PCR, wherein quantifying the expression level comprises obtaining a cycle threshold during quantitative PCR.

Provided herein is a process or method for computing prediction probabilities comprising: computing the probabilities as follows:

$${P}_{Group 4} = \frac{{E}_{Group 4}}{S} ;$$

$${P}_{Group 3} = \frac{{E}_{Group 3}}{S} ;$$

$${P}_{SHH} = \frac{{E}_{SHH }}{S} ;$$

$$and$$

$${P}_{WNT} = \frac{{E}_{WNT}}{S} ;$$

wherein,
S=Σₙ₌₁⁴E_{molecular subgroup}_{ₘ}
E_{molecular subgroup}_{ₘ}=e^{LP}^{_{molecular subgroup}}
LP_{molecular subgroup}_{ₘ}=β₀+Σₙ₌₁³¹(βₙ×ΔCT_{Gene}_{_{g}})
ΔCT_{Gene}_{_{g}}=CT_{HKG}−CT_{Gene}_{_{g}}
CT_{HKG}=\sqrt{(CT_{ACTB})²+(CT_{GAPDH})²};
βₙ is the coefficient that corresponds to a given gene;
   and assigning the sample to a class with the highest prediction probability.

In various embodiments, the βn coefficient for each gene is set forth in Table 3.

In various embodiments, the RNA sample is obtained from a subject having medulloblastoma tumor.

In some embodiments, the prediction probability is used to determine a course of therapy. In some embodiments, the therapy targets tumor-associated macrophages (TAM), such as inhibition of TAMs. In some embodiments, the therapy comprises inhibition of CSF1R inhibitor.

In various embodiments, subjects stratified into the SHH subgroup of medulloblastoma are prescribed decreased radiation therapy or no radiation therapy.

In various embodiments, subject stratified into Group 3 or Group 4 subgroup of medulloblastoma are prescribed normal radiation therapy or increased radiation therapy.

Also provided herein are computer systems comprising one or more processors wherein the computer system executes a software application implementing the process for computing prediction probabilities of a subject with medulloblastoma belonging to a specific subgroup.

Further provided herein is an article comprising one or more non-transitory machine-readable media storing instructions operable to cause one or more machines to perform operations, wherein the operations comprise implementing the process for computing prediction probabilities of a subject with medulloblastoma belonging to a specific subgroup.

Also provided herein is a non-transitory computer readable recording medium including programmed instructions, wherein the instructions, when executed by a computer that includes a display unit for displaying the probability of a subtype/subgroup of medulloblastoma, causes the computer to execute the process for computing prediction probabilities of a subject with medulloblastoma belonging to a specific subgroup.

Further provided herein is non-transitory computer readable media comprising instructions executable by one or more processors that when executed by one or more processors cause the one or more processors to perform the process for computing prediction probabilities of a subject with medulloblastoma belonging to a specific subgroup.

### BRIEF DESCRIPTION OF THE DRAWINGS

Exemplary embodiments are illustrated in referenced figures. It is intended that the embodiments and figures disclosed herein are to be considered illustrative rather than restrictive.

FIGS. 1A-1C depicts in accordance with various embodiments of the invention, FIG. 1A separation of 168 samples into 4 molecular subgroups (WNT, SHH, Group 3 and Group 4) using principal component analysis of 369 highly variable genes of the HuEx array data. FIG. 1B HuEx expression levels of CD163, a macrophage specific marker, showed highest expression in SHH subgroup of tumors. FIG. 1C Heatmap of gene expression levels of top 7 differentially expressed inflammation-related genes compared across molecular subgroups. Heatmap colors reflect expression values (log 2). Molecular group and histology of tumors are indicated by the color codes.

FIGS. 2A-2D depicts in accordance with various embodiments of the invention, FIG. 2A separation of medulloblastoma samples (n=83) using the first two principal components of the genes represented in the TLDA 31-gene signature. FIG. 2B The RT-PCR based TLDA assay validated HuEx results demonstrating significant increase in expression of CD163 and FIG. 2C CSF1R in the SHH subgroup compared to Group 3 and Group 4 subgroups. FIG. 2D CD163 expression was correlated with CSF1R (Pearson r=0.64, p<0.001).

FIGS. 3A-3E depicts in accordance with various embodiments of the invention, evidence of TAMs across medulloblastoma subgroups. Representative CD163 IHC images in tumor samples from FIG. 3A SHH subgroup with desmoplastic histology, FIG. 3B SHH with classic histology, FIG. 3C Group 3 subgroup, and FIG. 3D Group 4 subgroup. FIG. 3E Average CD163 IHC score is significantly higher in SHH tumors compared to Group 3 or Group 4 subgroups (p<0.0001 respectively).

FIGS. 4A-4D depicts in accordance with various embodiments of the invention, representative IHC images of tumors stained with anti-Ki-67 antibody in FIG. 4A SHH subgroup with desmoplastic histology, FIG. 4B SHH with classic histology, FIG. 4C Group 3 subgroup, and FIG. 4D Group 4 subgroup. Increased cell proliferation in the inter-nodular areas corresponded to the presence of macrophages in the SHH subgroup with desmoplastic histology.

FIG. 5 depicts in accordance with various embodiments of the invention, overview of samples studies and 31-gene signature development.

FIGS. 6A-6C depicts in accordance with various embodiments of the invention, non-negative matrix factorization (NMF) analysis of the combined cohort of medulloblastoma samples (n=168) FIG. 6A Cophenetic correlation coefficient based on 2000 clustering runs for 2-10 clusters utilizing genes obtained at varying coefficients of variation; the data demonstrate that the highest cophenetic correlation coefficient was obtained at 4 clusters with 369 genes. FIG. 6B NMF consensus heatmap of 4 subgroups using the dataset containing 369 genes with high coefficient of variation. FIG. 6C Silhouette plot for identification of outliers. Outliers (n=31) were defined as samples with a silhouette width <0.15.

FIG. 7 depicts in accordance with various embodiments of the invention, HuEx expression levels of CSF1R are significantly higher in SHH tumors compared to Group 3 or Group 4 subgroups (p<0.0001 respectively).

FIGS. 8A-8B depicts in accordance with various embodiments of the invention, progression free survival (PFS) and overall survival (OS) analyses using Kaplan-Meier plots and log rank tests for samples analyzed using TLDA 31-gene signature FIG. 8A PFS by molecular subgroup. FIG. 8B OS by molecular subgroup.

FIG. 9 depicts in accordance with various embodiments of the invention, TLDA expression levels of CD163 are similar among histologic subtypes of SHH medulloblastomas.

FIGS. 10A-10B depicts in accordance with various embodiments of the invention, progression free survival (PFS) and overall survival (OS) analyses using Kaplan-Meier plots and log rant tests for SHH tumors based on median TLDA expression of CD163. FIG. 10A PFS of high- vs low-expressers. FIG. 10 B OS of high- vs low-expressers. Children who died of treatment-related toxicity without evidence of disease progression are excluded from the analyses (n=3).

FIG. 11 depicts in accordance with various embodiments of the invention, additional representative (2 per group) of CD163 IHC images of SHH with desmoplastic histology, SHH with classic histology, Group 3 and Group 4 tumors.

FIG. 12 depicts in accordance with various embodiments of the invention, representative IHC images of SHH tumors with desmoplastic histology stained with anti-Ki-67 antibody demonstrating increased cell proliferation in internodular areas corresponding to the presence of macrophages.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N. Y. 2006); Smith, March's Advanced Organic Chemistry Reactions, Mechanisms and Structure 7^{th} ed., J. Wiley & Sons (New York, N. Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N. Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 March 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of a tumor, delay or slowing of a tumor, and amelioration or palliation of symptoms associated with a tumor.

“Disorders”, “diseases”, “conditions” and “disease conditions,” as used herein may include, but are in no way limited to any form of malignant neoplastic cell proliferative disorders or diseases. Examples of such disorders include but are not limited to cancer and tumor.

As used herein, the term “administering,” refers to the placement an agent as disclosed herein into a subject by a method or route which results in at least partial localization of the agents at a desired site. “Route of administration” may refer to any administration pathway known in the art, including but not limited to aerosol, nasal, oral, transmucosal, transdermal, parenteral, enteral, topical or local. “Parenteral” refers to a route of administration that is generally associated with injection, including intracranial, intraventricular, intrathecal, epidural, intradural, intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

The term “sample” or “biological sample” as used herein denotes a sample taken or isolated from a biological organism, e.g., a tumor sample from a subject. Exemplary biological samples include, but are not limited to, cheek swab; mucus; whole blood, blood, serum; plasma; urine; saliva; semen; lymph; fecal extract; sputum; other body fluid or biofluid; cell sample; tissue sample; tumor sample; and/or tumor biopsy etc. The term also includes a mixture of the above-mentioned samples. The term “sample” also includes untreated or pretreated (or pre-processed) biological samples. In some embodiments, a sample can comprise one or more cells from the subject. In some embodiments, a sample can be a tumor cell sample, e.g. the sample can comprise cancerous cells, cells from a tumor, and/or a tumor biopsy.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., medulloblastoma) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition can be a subject suspected of having that condition, diagnosed as having that condition, already treated or being treated for that condition, not treated for that condition, or at risk of developing that condition.

The term “statistically significant” or “significantly” refers to statistical evidence that there is a difference. It is defined as the probability of making a decision to reject the null hypothesis when the null hypothesis is actually true. The decision is often made using the p-value.

As used herein, “variants” can include, but are not limited to, those that include conservative amino acid mutations, SNP variants, splicing variants, degenerate variants, and biologically active portions of a gene. A “degenerate variant” as used herein refers to a variant that has a mutated nucleotide sequence, but still encodes the same polypeptide due to the redundancy of the genetic code.

The term “functional” when used in conjunction with “equivalent”, “analog”, “derivative” or “variant” or “fragment” refers to an entity or molecule which possess a biological activity that is substantially similar to a biological activity of the entity or molecule of which it is an equivalent, analog, derivative, variant or fragment thereof.

Medulloblastoma in children can be categorized into at least four molecular subgroups, offering the potential for targeted therapeutic approaches to reduce treatment related morbidities. Little is known about the role of tumor microenvironment in medulloblastoma or its contribution to these molecular subgroups. Tumor microenvironment has been shown to be an important source for therapeutic targets in both adult and pediatric neoplasms. In this study, we investigated the hypothesis that expression of genes related to tumor-associated macrophages (TAMs) correlates with the medulloblastoma molecular subgroups and contributes to a diagnostic signature.

A TaqMan™ Low-Density-Array assay was created that uses a panel of 31 genes chosen for their ability to distinguish the subtypes of medulloblastoma, wherein the subtypes are Group 3, Group 4, SHH and WNT. The 31 genes are βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC, βWIF1. Using the methods described herein, probabilities may be assigned as to the subtype of medulloblastoma in a subject, allowing selection of more targeted therapies.

Gene expression profiling using Human Exon Array (n=168) was analyzed to identify medulloblastoma molecular subgroups and expression of inflammation-related genes. Expression of 45 tumor-related and inflammation-related genes was analyzed in 83 medulloblastoma samples to build a gene signature predictive of molecular subgroups. TAMs in medulloblastomas (n=54) comprising the four molecular subgroups were assessed by immunohistochemistry (IHC).

A 31-gene medulloblastoma subgroup classification score inclusive of TAM-related genes (CD163, CSF1R) was developed with a misclassification rate of 2%. Tumors in the Sonic Hedgehog (SHH) subgroup had increased expression of inflammation-related genes and significantly higher infiltration of TAMs than tumors in the Group 3 or Group 4 subgroups (p<0.0001 and p<0.0001, respectively). IHC data revealed a strong association between location of TAMs and proliferating tumor cells.

These data show that SHH tumors have a unique tumor microenvironment among medulloblastoma subgroups. The interactions of TAMs and SHH medulloblastoma cells may contribute to tumor growth revealing TAMs as a potential therapeutic target.

Accordingly, embodiments of the present invention provide a highly sensitive and specific method for determining subgroups of medulloblastoma so as to prescribe and/or administer therapies that specifically treat the medulloblastoma subtypw/subgroup. For example, in patients with SHH subgroup of medulloblastoma, therapies that inhibit TAM may be recommended and administered.

In an embodiment, the present invention provides a method of diagnosing a medulloblastoma subtype in a subject comprising, consisting of or essentially consisting of obtaining a sample from the subject and assaying the sample to determine the expression profile of any one or more of βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC, βWIF1, or a combination thereof. The method further comprises computing the prediction probabilities of the sample belonging to the subgroup of medulloblastoma and assigning the sample to a subgroup with the highest prediction probability. In an embodiment, the subgroups of medulloblastoma include Group 3, Group 4, SHH and WNT. In various embodiments, the sample is an RNA sample. In various embodiments, the sample is obtained from the medulloblastoma tumor in the subject and RNA is extracted from the tumor sample.

In an embodiment, the present invention provides a method of diagnosing a medulloblastoma subtype in a subject comprising, consisting of or essentially consisting of obtaining a sample from the subject and assaying the sample to determine the expression profile of βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC and βWIF1. The method further comprises computing the prediction probabilities of the sample belonging to the subgroup of medulloblastoma and assigning the sample to a subgroup with the highest prediction probability. In an embodiment, the subgroups of medulloblastoma include Group 3, Group 4, SHH and WNT. In various embodiments, the present invention provides a method for classifying a cancer in a subject. In various embodiments, the sample is an RNA sample. In various embodiments, the sample is obtained from the medulloblastoma tumor in the subject and RNA is extracted from the tumor sample.

Various embodiments of the present invention provides a process for determining the likelihood that a subject has a specific subtype/subgroup of medulloblastoma, comprising: providing a first composition comprising a plurality of isolated nucleic acids probes; contacting the first composition to an RNA sample from a mammalian subject desiring a determination of a subgroup of medulloblastoma, to produce one or more cDNA molecules; providing a second composition comprising one or more isolated nucleic acids probes comprising a sequence capable of hybridizing to one or more nucleic acids selected from the group of genes consisting of βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC, βWIF1 or a combination thereof; contacting the second composition with the one or more cDNA molecules to amplify the one or more cDNA molecules; quantifying the expression level of the one or more genes to determine the subgroup of medulloblastoma to which the sample belongs; and computing the prediction probabilities of the sample belonging to the subgroup of medulloblastoma. In various embodiments, the sample is an RNA sample. In various embodiments, the sample is obtained from the medulloblastoma tumor in the subject and RNA is extracted from the tumor sample.

Various embodiments of the present invention provides a process to detect medulloblastoma in a subject in need by detecting the medulloblastoma subtype/subgroup, comprising: providing a first composition comprising a plurality of isolated nucleic acids probes; contacting the first composition to an RNA sample from a mammalian subject desiring a determination of a subgroup of medulloblastoma, to produce one or more cDNA molecules; providing a second composition comprising a plurality of nucleic acids probes comprising a sequences capable of hybridizing to nucleic acids for βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC and βWIF1; contacting the second composition with the one or more cDNA molecules to amplify the one or more cDNA molecules; quantifying the expression level of the one or more genes to determine the subgroup of medulloblastoma to which the sample belongs; and computing the prediction probabilities of the sample belonging to the subgroup of medulloblastoma. In various embodiments, the sample is an RNA sample. In various embodiments, the sample is obtained from the medulloblastoma tumor in the subject and RNA is extracted from the tumor sample.

Various embodiments of the present invention provide a process to stratify subject into subgroups of medulloblastoma so as to optimize treatment in the subject. The process comprises: providing a first composition comprising a plurality of isolated nucleic acids probes; contacting the first composition to an RNA sample from a mammalian subject desiring a determination of a subgroup of medulloblastoma, to produce one or more cDNA molecules; providing a second composition comprising a plurality of nucleic acids probes comprising a sequences capable of hybridizing to nucleic acids for βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC and βWIF1; contacting the second composition with the one or more cDNA molecules to amplify the one or more cDNA molecules; quantifying the expression level of the one or more genes to determine the subgroup of medulloblastoma to which the sample belongs; and computing the prediction probabilities of the sample belonging to the subgroup of medulloblastoma. In various embodiments, the sample is an RNA sample. In various embodiments, the sample is obtained from the medulloblastoma tumor in the subject and RNA is extracted from the tumor sample.

In some embodiments, patients with SHH subgroup of medulloblastoma are not prescribed and/or administered radiation therapy. In some embodiments, patients with SHH subgroup of medulloblastoma are prescribed and/or administered reduced/decreased doses of radiation therapy. Reduced/decreased doses of radiation therapy will be apparent to a person of skill in the art. In exemplary embodiments, in subjects with SHH subgroup of medulloblastoma, radiation therapy is decreased by 90%, 80%, 70%, 60%, 50%, 40%, 30%, 20%, 10% or 5% relative to radiation therapy administered in subject with cancer (for example, subjects with brain cancer). In exemplary embodiments, in subjects with SHH subgroup of medulloblastoma, radiation therapy is decreased by 5-fold, 10-fold, 20-fold, 30-fold, 40-fold, 50-fold, 60-fold, 70-fold, 70-fold or 90-fold relative to radiation therapy administered in subject with cancer (for example, subjects with brain cancer).

In some embodiments, patients with WNT subgroup of medulloblastoma are not prescribed and/or administered radiation therapy. In some embodiments, patients with WNT subgroup of medulloblastoma are prescribed and/or administered reduced/decreased doses of radiation therapy. Reduced/decreased doses of radiation therapy will be apparent to a person of skill in the art. In exemplary embodiments, in subjects with WNT subgroup of medulloblastoma, radiation therapy is decreased by 90%, 80%, 70%, 60%, 50%, 40%, 30%, 20%, 10% or 5% relative to radiation therapy administered in subject with cancer (for example, subjects with brain cancer). In exemplary embodiments, in subjects with WNT subgroup of medulloblastoma, radiation therapy is decreased by 50% or 100% relative to radiation therapy administered in subject with cancer (for example, subjects with brain cancer), or delayed for 1-2 years after diagnosis. In exemplary embodiments, in subjects with WNT subgroup of medulloblastoma, radiation therapy is decreased by 5-fold, 10-fold, 20-fold, 30-fold, 40-fold, 50-fold, 60-fold, 70-fold, 70-fold or 90-fold relative to radiation therapy administered in subject with cancer (for example, subjects with brain cancer).

In some embodiments, patients with Group 3 and/or Group 4 subgroup of medulloblastoma are prescribed and/or administered normal or increased dosages of radiation therapy. Increased doses of radiation therapy will be apparent to a person of skill in the art. In exemplary embodiments, in subjects with Group 3 and/or Group 4 subgroup of medulloblastoma, radiation therapy is increased by 5%, 10%, 20%, 30%, 40%, 50%, 60%, 70%, 80% or 90% relative to radiation therapy administered in subject with cancer (for example, subjects with brain cancer). In exemplary embodiments, in subject with Group 3 and/or Group 4 subgroup of medulloblastoma, radiation therapy is increased by 5-fold, 10-fold, 20-fold, 30-fold, 40-fold, 50-fold, 60-fold, 70-fold, 70-fold or 90-fold relative to radiation therapy administered in subject with cancer (for example, subjects with brain cancer). In exemplary embodiments, in subjects with Group 3 and/or Group 4 subgroup of medulloblastoma, radiation therapy is increased by 1.5 fold relative to radiation therapy administered in subject with cancer (for example, subjects with brain cancer).

In various embodiments, the subject is a child with medulloblastoma. In various embodiments, the subgroup/subtype of medulloblastoma is Group 3, Group 4, WNT and SHH. In various embodiments, the prediction probabilities are computed using the formulas in Equations 1-9 as described herein. The co-efficients for each of the 31 genes in each of the subgroups/subtypes are described in Table 3. The β₀ intercept of each subgroup is shown in the bottom row of Table 3.

In various embodiments, quantifying the expression of any one or more of βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC, βWIF1, or a combination thereof comprises employing quantitative amplification. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and sybr green. Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc. In an exemplary embodiment, methods described in Geiss et al. (Direct multiplexed measurement of gene expression with color-coded probe pairs. Nat Biotechnol. 2008 March; 26(3):317-25) may be used with the methods described herein.

In various embodiments, the present invention provides a composition, comprising one or more isolated nucleic probes comprising sequences capable of hybridizing to any one or more of βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC, βWIF1, or a combination thereof. In various embodiments, the present invention provides a composition, comprising isolated nucleic probes comprising sequences capable of hybridizing to βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC and βWIF1. In various embodiments, the present invention also provides an array, comprising, consisting of or essentially consisting of a substrate having any of the above-described compositions.

### Computation of Prediction Probabilities for 31-Gene Medulloblastoma Predictor:

Described herein are coefficients used for computing classification probabilities for the Group 3, Group 4, SHH, and WNT classes of medulloblastoma (MB) based on the recently developed 31-gene predictor. Column one of Table 3 provides the coefficient (13) that corresponds to a given gene in the 31-gene signature (alphabetical order by gene name). The last row in Table 3 is the intercept constant (β₀) value. Each column gives the coefficient values that correspond to each MB molecular subgroup. The housekeeping (HKG) genes ACTB and GAPDH were used to normalize expression values of a given gene in the signature. The geometric mean of the two HKGs was used to derive CT_{HKG} (Equation 1), wherein CT is the cycle threshold. ΔCT of a given gene (Gene_{g}) was normalized to CTHKG per Equation 2.

CT_{HKG}=\sqrt{(CT_{ACTB})²+(CT_{GAPDH})²} Equation 1.

ΔCT_{Gene}_{_{g}}=CT_{HKG}−CT_{Gene}_{_{g}} Equation 2.

Computation of prediction probabilities for 31-gene medulloblastoma predictor include:

For each molecular subgroup column (Group 3, Group 4, SHH, WNT) in Table 3:
(i) Compute the sumproduct of the ΔCT_{Gene}_{_{g}} and its corresponding coefficient value βₙ and add the constant (β₀) to this. The result is termed the linear predictor (LP) (Equation 3). m refers to the molecular subgroup which may be Group 3, Group 4, SHH or WNT. Gene (g) refers to one of the signature genes in Table 3.

LP_{molecular subgroup}_{ₘ}=β₀+Σₙ₌₁³¹(βₙ×ΔCT_{Gene}_{_{g}}) Equation 3.
(ii) For each of the molecular subgroup LP values (i.e., LP_{Group3}, LP_{Group4}, LP_{SHH}, LP_{WNT}) compute the exponentiated quantity of the LP for each molecular subgroup (Equation 4).

E_{molecular subgroup}_{ₘ}=e^{LP}^{_{molecular subgroup}} Equation 4.
(iii) Compute the sum of the four E_{molecular subgroup} (Equation 5).

S=Σₙ₌₁⁴E_{molecular subgroup}_{ₘ} Equation 5.
(iv) Compute the prediction probabilities of a sample belonging to each molecular subgroup as:

$$\begin{matrix}{P}_{Group 4} = \frac{{E}_{Group 4}}{S} & Equation 6 \\ {P}_{Group 3} = \frac{{E}_{Group 3}}{S} & Equation 7 \\ {P}_{SHH} = \frac{{E}_{SHH }}{S} & Equation 8 \\ {P}_{WNT} = \frac{{E}_{WNT}}{S} & Equation 9\end{matrix}$$

(v) The patient is assigned to the molecular subgroup with the highest prediction probability.

### Treatment Methods

In another embodiment, the present invention provides a method for treating a subject with medulloblastoma. The method includes diagnosing the subtype of medulloblastoma in the subject by the methods set forth herein and prescribing appropriate therapy. In an embodiment, in a subject with SHH subgroup of medulloblastoma, inhibition of TAM, for example through inhibition of CSF1R is prescribed and/or administered. In an embodiment, the subject in SHH subgroup of medulloblastoma is prescribed decreased radiation therapy or no radiation therapy. In an embodiment, subjects in Group 3 or Group 4 subgroup of medulloblastoma are prescribed radiation therapy. In an embodiment, subjects in Group 3 or Group 4 subgroup of medulloblastoma, who are already receiving radiation therapy, are prescribed increased radiation therapy. The radiation therapy dosages associated with increased or decreased radiation therapy will be apparent to a person of skill in the art.

In some embodiments, provided herein are methods for treating subjects with Group 3 subgroup of medulloblastoma. The methods comprise, consist of or essentially consist of diagnosing the subtype/subgroup of medulloblastoma in the subject by the methods set forth herein and prescribing a therapeutic agent that is specific for treating subjects with Group 3 subgroup of medulloblastoma to the subject with Group 3 subgroup of medulloblastoma. The method further comprises administering to the subject an effective amount of the therapeutic agent to the subject so as to treat Group 3 subgroup/subtype of medulloblastoma in the subject.

In some embodiments, provided herein are methods for treating subjects with Group 4 subgroup of medulloblastoma. The methods comprise, consist of or essentially consist of diagnosing the subtype/subgroup of medulloblastoma in the subject by the methods set forth herein and prescribing a therapeutic agent that is specific for treating subjects with Group 4 subgroup of medulloblastoma to the subject with Group 4 subgroup of medulloblastoma. The method further comprises administering to the subject an effective amount of the therapeutic agent to the subject so as to treat Group 4 subgroup/subtype of medulloblastoma in the subject.

In some embodiments, provided herein are methods for treating subject with SHH subgroup of medulloblastoma. The methods comprise, consist of or essentially consist of diagnosing the subtype/subgroup of medulloblastoma in the subject by the methods set forth herein and prescribing a therapeutic agent that is specific for treating subjects with SHH subgroup of medulloblastoma to the subject with SHH subgroup of medulloblastoma. The method further comprises administering to the subject an effective amount of the therapeutic agent to the subject so as to treat SHH subgroup/subtype of medulloblastoma in the subject.

In some embodiments, provided herein are methods for treating subject with WNT subgroup of medulloblastoma. The methods comprise, consist of or essentially consist of diagnosing the subtype/subgroup of medulloblastoma in the subject by the methods set forth herein and prescribing a therapeutic agent that is specific for treating subjects with WNT subgroup of medulloblastoma to the subject with WNT subgroup of medulloblastoma. The method further comprises administering to the subject an effective amount of the therapeutic agent to the subject so as to treat WNT subgroup/subtype of medulloblastoma in the subject.

In various embodiments, the subject is a human. In various embodiments, the subject is a mammalian subject including but not limited to human, monkey, ape, dog, cat, cow, horse, goat, pig, rabbit, mouse and rat. In various embodiments, the biological sample comprises a cell, neuron, glia, brain cell, spinal cord cell, brain neuron, brain glia, spinal cord neuron, or spinal cord glia, or a combination thereof. In some embodiments, the biological sample comprises a tumor cell or tissue. In some embodiments, the biological sample comprises a tumor biopsy or sample.

Typical dosages of an effective amount of the therapeutic can be in the ranges recommended by the manufacturer where known therapeutic molecules or compounds are used, and also as indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models. In various embodiments, the therapeutic may be administered once a day (SID/QD), twice a day (BID), three times a day (TID), four times a day (QID), or more, so as to administer an effective amount of the therapeutic to the subject, where the effective amount is any one or more of the doses described herein.

In various embodiments, the therapeutic is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m2, or a combination thereof. In various embodiments, the therapeutic is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg, or a combination thereof. In various embodiments, the therapeutic is administered once, twice, three or more times. In various embodiments, the therapeutic is administered about 1-3 times per day, 1-7 times per week, 1-9 times per month, or 1-12 times per year. In various embodiments, the therapeutic is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. Here, “mg/kg” refers to mg per kg body weight of the subject, and “mg/m2” refers to mg per m2 body surface area of the subject. In certain embodiments, the therapeutic is administered to a human.

In various embodiments, the effective amount of the therapeutic is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 μg/kg/day, or a combination thereof. In various embodiments, the effective amount of the therapeutic is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 μg/m2/day, or a combination thereof. In various embodiments, the effective amount of the therapeutic is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg/day, or a combination thereof. In various embodiments, the effective amount of the therapeutic is any one or more of about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/m2/day, or a combination thereof. Here, “m/kg/day” or “mg/kg/day” refers to μg or mg per kg body weight of the subject per day, and “m/m2/day” or “mg/m2/day” refers to μg or mg per m2 body surface area of the subject per day.

In some embodiments, the therapeutic may be administered at the prevention stage of a condition (i.e., when the subject has not developed the condition but is likely to or in the process to develop the condition). In other embodiments, the therapeutic may be administered at the treatment stage of a condition (i.e., when the subject has already developed the condition). As a non-limiting example, the target condition is medulloblastoma (for example, a subtype of medulloblastoma as described herein). In this exemplar situation, the patient may be treated with the methods described herein when the patient has not yet developed medulloblastoma, or is likely to develop medulloblastoma, or is in the process of developing medulloblastoma, or has already developed medulloblastoma.

In accordance with the invention, the therapeutic may be administered using the appropriate modes of administration, for instance, the modes of administration recommended by the manufacturer for each of the therapeutic. In accordance with the invention, various routes may be utilized to administer the therapeutic of the claimed methods, including but not limited to aerosol, nasal, oral, transmucosal, transdermal, parenteral, enteral, topical, local, implantable pump, continuous infusion, capsules and/or injections. In various embodiments, the therapeutic is administered intracranially, intraventricularly, intrathecally, epidurally, intradurally, topically, intravascularly, intravenously, intraarterially, intratumorally, intramuscularly, subcutaneously, intraperitoneally, intranasally, or orally.

In various embodiments, the therapeutic is provided as a pharmaceutical composition. In various embodiments, the composition is formulated for via any route of administration, including but not limited to intracranial, intraventricular, intrathecal, epidural, intradural, topical, intravascular, intravenous, intraarterial, intratumoral, intramuscular, subcutaneous, intraperitoneal, intranasal or oral administration. Methods for these administrations are known to one skilled in the art. Preferred pharmaceutical compositions will also exhibit minimal toxicity when administered to a mammal.

### Systems and Computers

In certain embodiments, the methods of the invention implement a computer program to calculate expression of genes of interest and calculate prediction probabilities that a subject has a specific subtype of medulloblastoma. For example, a computer program can be used to perform the algorithms described herein. A computer system can also store and manipulate data generated by the methods of the present invention which comprises a plurality of hybridization signal changes/profiles during approach to equilibrium in different hybridization measurements and which can be used by a computer system in implementing the methods of this invention. In certain embodiments, a computer system receives probe hybridization data; (ii) stores probe hybridization data; and (iii) computes the prediction probabilities of the sample belonging to a subgroup of medulloblastoma. In certain embodiments, such computer systems are also considered part of the present invention.

Numerous types of computer systems can be used to implement the analytic methods of this invention according to knowledge possessed by a skilled artisan in the bioinformatics and/or computer arts.

Several software components can be loaded into memory during operation of such a computer system. The software components can comprise both software components that are standard in the art and components that are special to the present invention (e.g., dCHIP software described in Lin et al. (2004) Bioinformatics 20, 1233-1240; CRLMM software described in Silver et al. (2007) Cell 128, 991-1002; Aroma Affymetrix software described in Richardson et al. (2006) Cancer Cell 9, 121-132. The methods of the invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.). In certain embodiments, the computer comprises a database for storage of hybridization signal profiles. Such stored profiles can be accessed and used to calculate the prediction probabilities of the sample belonging to a specific subgroup of medulloblastoma.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

Embodiments of the invention can be described through functional modules, which are defined by computer executable instructions recorded on computer readable media and which cause a computer to perform method steps when executed. In various embodiments, the computer executable instructions comprise, consist of or essentially consist of executing the methods described herein to compute the prediction probabilities of a sample belonging to the subgroup of medulloblastoma. The modules are segregated by function, for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks), BLU-RAY disc or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the invention include for example, at a measuring module, a storage module, a comparison module, and an output module. The functional modules can be executed on one, or multiple, computers, or by using one, or multiple, computer networks. The measuring module has computer executable instructions to provide e.g., expression information in non-transitory computer readable form.

The measuring module can comprise any system for detecting the expression patterns of βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC, βWIF1 or a combination thereof. Such systems can include DNA microarrays, RNA expression arrays, any ELISA detection system and/or any Western blotting detection system.

The information determined in the determination system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon expression level or protein level information. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In one embodiment the reference data stored in the storage module to be read by the comparison module is, for example, expression data obtained from a population of non-cancer subjects, a population of cancer subjects, or expression data obtained from the same subject at a prior time point using the measuring module.

The “comparison module” can use a variety of available software programs and formats for the comparison operative to compare expression data determined in the measuring module to reference samples and/or stored reference data. In one embodiment, the comparison module is configured to use pattern recognition techniques to compare information from one or more entries to one or more reference data patterns. The comparison module may be configured using existing commercially-available or freely-available software for comparing patterns, and may be optimized for particular data comparisons that are conducted. The comparison module provides computer readable information related to the expression patterns of βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC, βWIF1 or a combination thereof in an individual, efficacy of treatment in an individual, and/or method for treating an individual.

The comparison module, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets. An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content-based in part on the comparison result that may be stored and output as requested by a user using an output module.

The content based on the comparison result, may be an expression value compared to a reference showing the susceptibility/adequate response or nonsusceptibility/non-adequate response from standard, conventional or certain therapy.

In various embodiments of the invention, the content based on the comparison result is displayed on a computer monitor. In various embodiments of the invention, the content based on the comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

The present invention therefore provides for systems (and computer readable media for causing computer systems) to perform methods for selecting treatment of cancer in an individual. As used herein, “selecting treatment” refers to selecting, choosing or prescribing a cancer treatment for the individual, or instructing or directing the individual to receive a cancer treatment.

Systems and computer readable media described herein are merely illustrative embodiments of the invention for detecting the expression patterns of βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC, βWIF1 or a combination thereof and computation of prediction probabilities of the sample belonging to a subgroup of medulloblastoma, and are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

### Example 1

### Experimental Methods

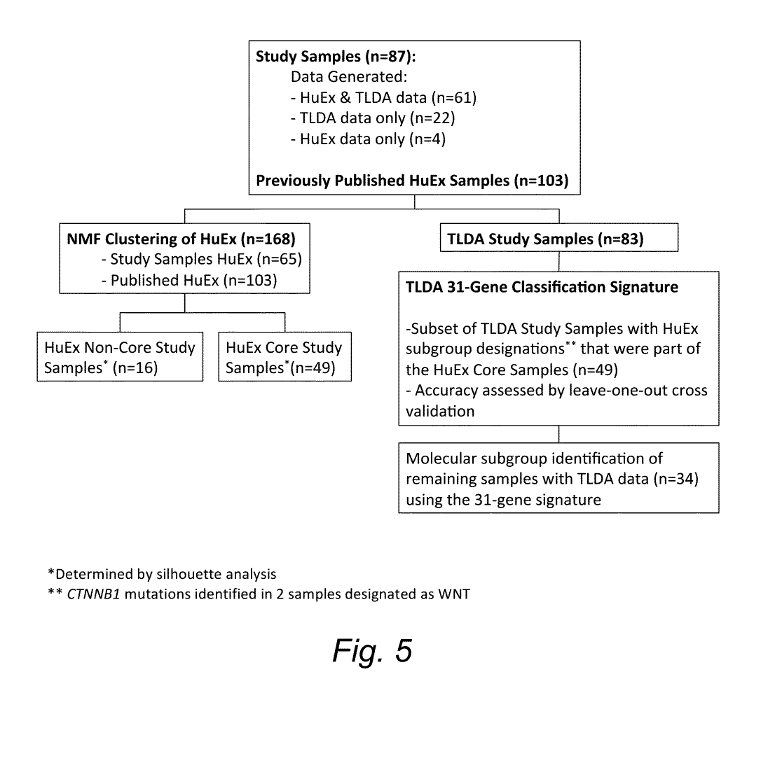

Samples were collected from patients with medulloblastoma (primary samples n=85, relapse samples n=2) treated at Children's Hospital Los Angeles (CHLA) (Los Angeles, Calif.) or Cincinnati Children's Hospital Medical Center (CCHMC) (Cincinnati, Ohio) between 1989 and 2012 with available adequate fresh frozen tissue for evaluation. All samples underwent pathologic review by two neuropathologists to confirm the diagnosis. The patient and tumor characteristics are provided in Table 4. Sixty-five samples underwent Affymetrix Human Exon 1.0 ST Array (HuEx) analysis. The data from these 65 HuEx data were analyzed in combination with data from a cohort of 103 samples (FIG. 5 and Table 6). Additional 36 samples and a subset of HuEx samples with sufficient RNA (n=47 of 65) were analyzed using a custom medulloblastoma-specific TLDA assay (total n=83, Table 5). The details of analyses performed on the HuEx microarray and the custom TLDA assay data are provided herein. In brief, the molecular subgroups were identified in an unsupervised manner using the HuEx data and performing 1000 runs of non-negative matrix factorization (NMF) clustering on several subsets of genes with high coefficient of variation (Gaujoux R, Seoighe C. A flexible R package for nonnegative matrix factorization. BMC Bioinformatics; 2010; 11:367). Silhouette analysis (Rousseeuw P. Silhouettes: A graphical aid to the interpretation and validation of cluster analysis. J Comput Appl Math. 1987; 20:53-65) was used to identify samples with high silhouette width for a given subgroup's cluster, indicating higher similarity to their own subgroup than to any other molecular subgroup (Table 6). These samples with large silhouette width along with two samples designated as WNT group based on mutational analysis of CTNNB1 (exon 3) gene (Eberhart C G, et al. Nuclear localization and mutation of beta-catenin in medulloblastomas. J Neuropathol Exp Neurol. 2000; 59:333-7; Clifford S C, et al. Wnt/Wingless pathway activation and chromosome 6 loss characterize a distinct molecular sub-group of medulloblastomas associated with a favorable prognosis. Cell Cycle. 2006; 5:2666-70) were used as core samples or true positives in constructing and validating the TLDA signature. The molecular subgroup of the remaining samples was predicted using the TLDA signature. FIG. 5 provides a schematic outline of the experimental design and samples used for generating the TLDA signature.

Macrophages were identified using immunohistochemical (IHC) analysis of 54 of the 83 medulloblastoma samples for which molecular subgroups had been determined using an antibody directed against CD163 as previously described (Asgharzadeh S, et al. Clinical significance of tumor-associated inflammatory cells in metastatic neuroblastoma J Clin Oncol. 2012; 30:3525-32). Paraffin tissue section scores ranged from 0 to 3, with higher scores indicating a greater proportion of positive cells. Two neuropathologists independently scored all samples and the mean of the scores was used for further analyses. Twenty-three samples were also stained using an antibody against Ki-67 to assess association of macrophages and cell proliferation.

### Statistical Methods

Common statistical analyses including analysis of variance (ANOVA) with linear contrast, chi-squared, and Spearman rank correlation coefficient were used where appropriate and are indicated in the text. Statistical computations were performed using the R project (http://www.r-project.org) or Stata 11 (StataCorp. 2009. Stata Statistical Software: Release 11. College Station, Tex.: StataCorp LP).

### Identification of Molecular Subgroup Using HuEx Assay

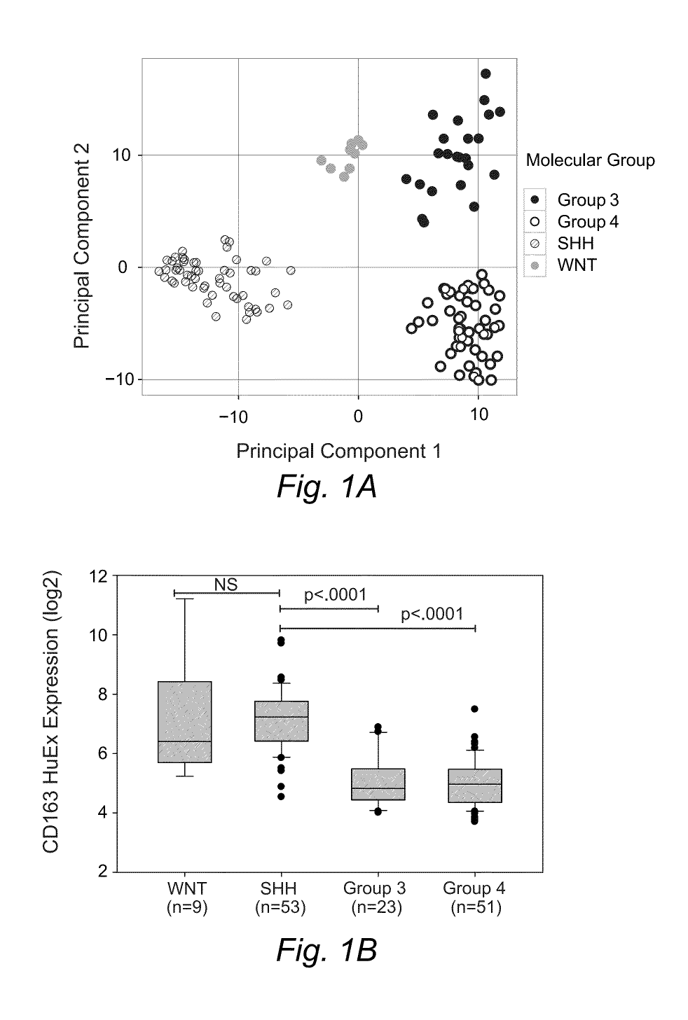

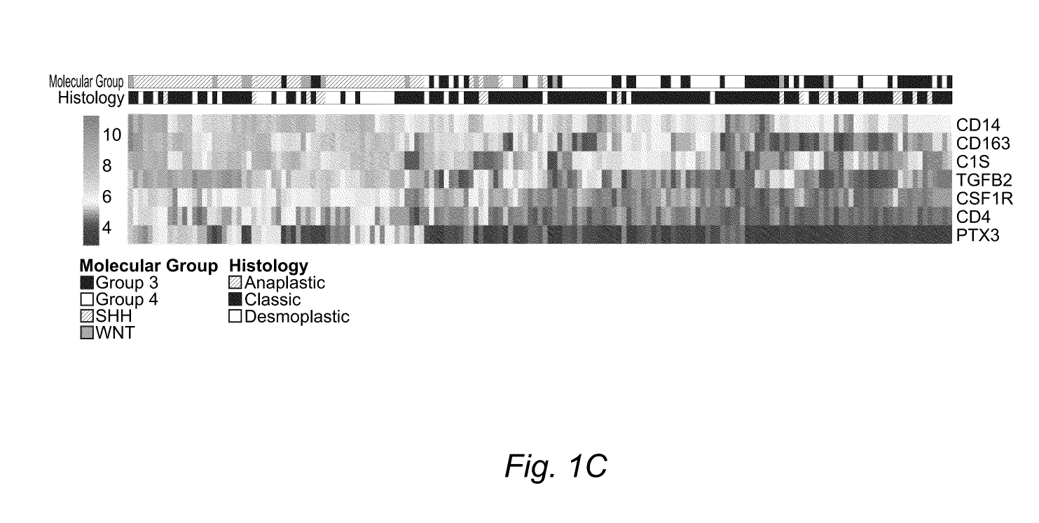

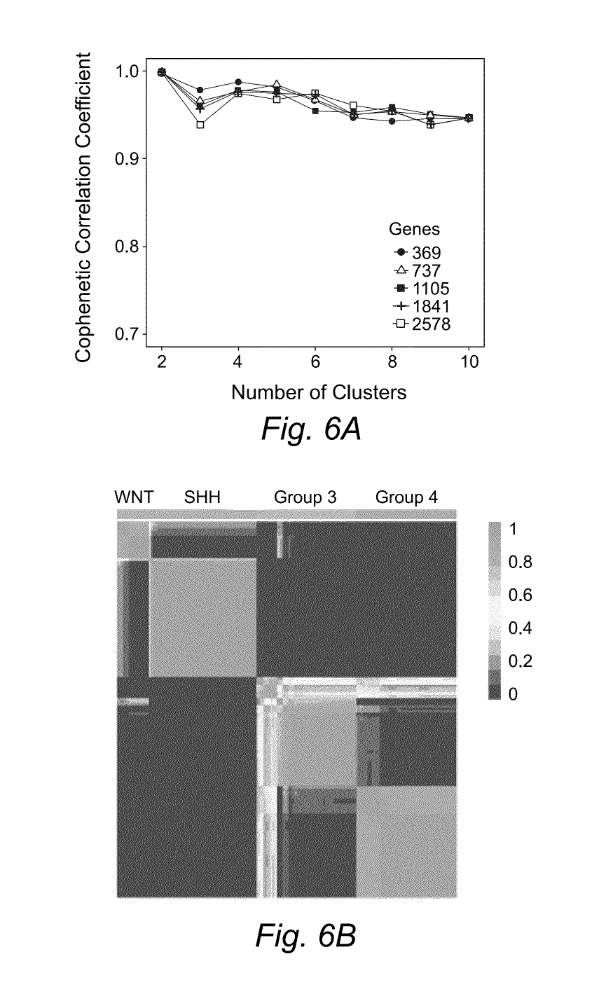

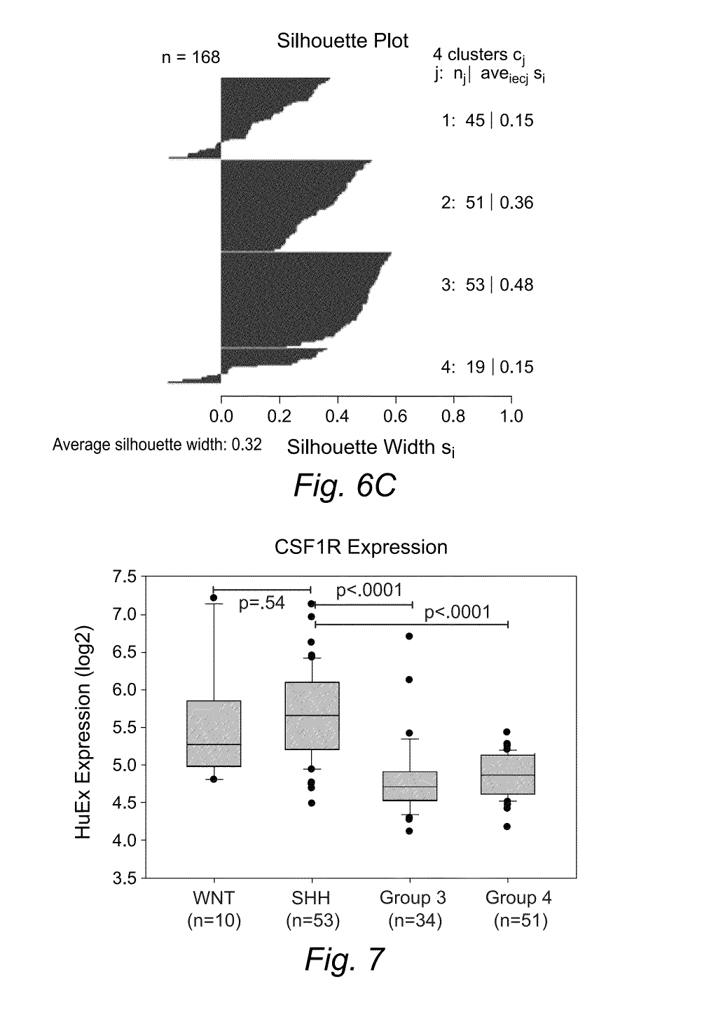

Molecular subgroups of medulloblastomas were identified using HuEx microarray data from 168 samples (65 study patients and 103 from previously published cohort) using an algorithm based on NMF (FIG. 1A and FIG. 6). There was an extremely high concordance (92%) between the molecular subgroup designation of the 103 patients previously published and results obtained with our combined analysis (Table 6), with majority of discordant findings occurring between Group 3 and Group 4. The patient characteristics and distribution of tumors among molecular subgroups for the 65 CHLA patients were similar to previous published reports (Table 6) (Kool M, et al. Integrated genomics identifies five medulloblastoma subtypes with distinct genetic profiles, pathway signatures and clinicopathological features. PLoS ONE. 2008; 3:e3088; Northcott P A, et al. Medulloblastoma Comprises Four Distinct Molecular Variants. J Clin Oncol. 2011; 29:1408-14).

### Patient Selection and Sample Preparation

All tumor specimens were obtained in accordance with the Research Ethics Board at Children's Hospital Los Angeles (CHLA) (Los Angeles, Calif.) and Cincinnati Children's Hospital Medical Center (Cincinnati, Ohio). A total of 87 medulloblastomas samples were obtained as surgically resected, fresh-frozen samples. In addition, we obtained paraffin embedded tissue from 56 of those samples.

### Immunohistochemical Studies

Sections of paraffin embedded tissue were obtained from patients treated at CHLA (n=56). The samples were cut and stained at CHLA using an antibody against CD163 (Leica Microsystems) with appropriate negative controls. Two sections were not evaluable due to poor tissue preservation. In total there were 54 samples included in the final analysis: SHH (n=22), WNT (n=2), Group 3 (n=9), Group 4 (n=21). The antibody staining was scored from 0 to 3 in increments of 1. The scoring system was proposed by AJ and reflects the percentage of macrophages that occupy the septal spaces between tumor cells. Two pathologists (AJ and FG) scored all of the specimens independently and blinded to the molecular grouping of samples. No sample had a divergent score of greater than one point and no consensus score was needed. The mean score for each sample was calculated for further analysis.

### Gene Expression Studies

### Sample Preparation

Total RNA from frozen tumor sections of the medulloblastoma samples was isolated for microarray analysis using TRIzol reagent at CHLA. The percentage of tumoral cells was evaluated by microscopic evaluation of hematoxylin- and eosin-stained sections. Only samples of tumors with ≧80% tumoral cells were included in this study. RNA quality for all samples was assessed at CHLA by Nanodrop spectrophotometry and RNA integrity number (RIN) using a Bioanalyzer 2100 (Agilent Technologies, Santa Clara, Calif.). Only the samples with RIN>6.5 underwent further processing by microarray or TLDA.

### Data Sources: Microarrays

### Human Exon Array Data

We used Affymetrix Human Exon 1.0 ST Array (HuEx array) to analyze medulloblastoma tumor samples collected from CHLA (n=65) and combined the expression data with that of a previously published cohort (Gene Expression Omnibus (GEO) GSE21140) (n=103) (Northcott P A, et al: Medulloblastoma Comprises Four Distinct Molecular Variants. J Clin Oncol 29:1408-1414, 2011).

### Summarization, Filtering and Normalization

The complete ‘full’ probeset regions (PSR) of HuEx array were normalized and summarized using Affymetrix Power Tools (APT ver. 1.12.0, Affymetrix, Palo Alto, Calif.) and subsequent gene summaries were derived from annotations built using hg19 version of the human genome. PSRs from the full dataset whose expression was less than the median of all PSRs and whose coefficient of variation (cv) was less than the median cv value of all the PSRs were excluded from further analyses. These filtered PSRs (approximately 10% of all PSRs) were deemed noncontributory to gene levels.

Transcript average values used for this study were derived from this filtered PSR set which were annotated as ‘core’ by Affymetrix annotation files. For cluster analyses, probe sets representing genes on chromosome Y were filtered out in order to avoid the gender based batch effect in the clustering algorithm.

### Removing Institutional Batch Effect

Analysis of variance (ANOVA) was used to remove batch effect related to institution origin of the datasets (Sick Kiids Hospital in Toronoto versus Children's Hospital Los Angeles). In brief, each PSR was used to fitting a model for each of the Human Exon array probeset and obtaining the residuals after removing the main institution effect. All ANOVA models adjusted for histology.

### Clustering

### Nonnegative Matrix Factorization

Nonnegative Matrix Factorization (NMF) was used to identify number of likely clusters in our dataset in an unsupervised fashion [Gaujoux R, Seoighe C: A flexible R package for nonnegative matrix factorization. BMC Bioinformatics 11:367, 2010]. NMF (Brunet approach) was utilized to extract relevant biological information from microarray data and uses Kullbach-Leibler divergence algorithm where it uses simple multiplicative updates to avoid numerical underflow (Seung D, Lee L: Algorithms for non-negative matrix factorization. Advances in neural information processing systems, 2001). The NMF package for the R statistical software was used to perform the clustering computations (Gaujoux R, Seoighe C: A flexible R package for nonnegative matrix factorization. BMC Bioinformatics 11:367, 2010). We applied NMF to different sets of highly variable genes in our HuEx datasets (ranging from ˜400 genes to 2500 genes) to identify cluster predictions for cluster sizes ranging from 2 to 9. Each NMF algorithm was run with 1000 iterations and using the Burnet algorithm. Based on the value of the cophenetic coefficients, our NMF predicted robust clusters starting at k=4 (FIG. 5).

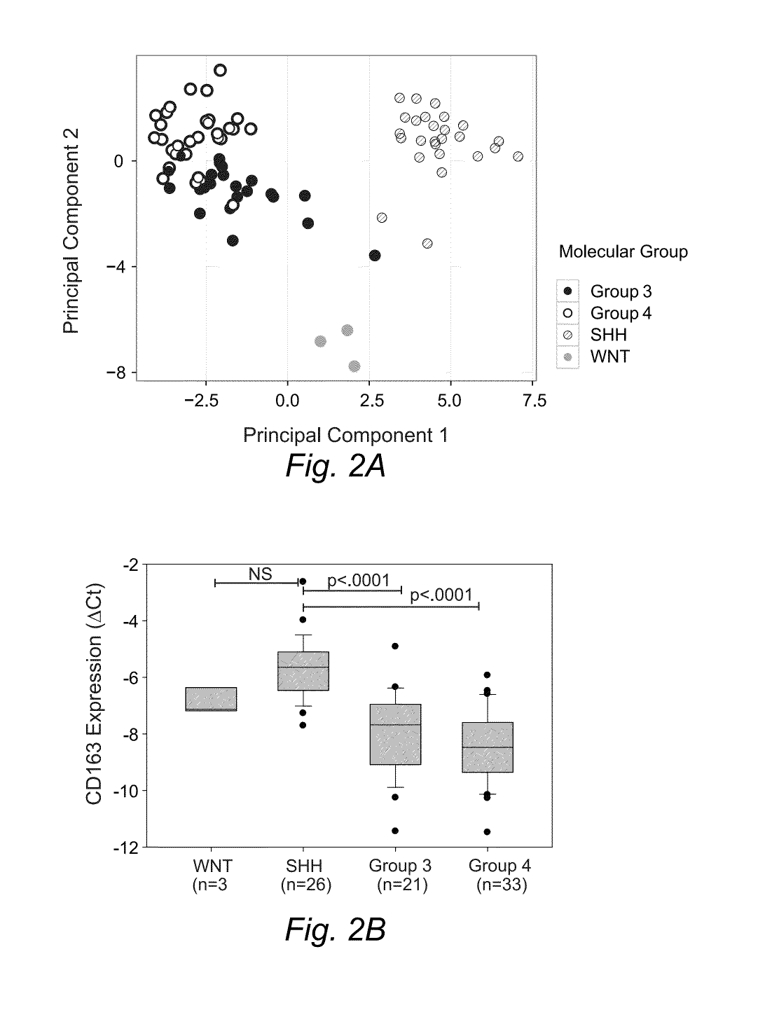

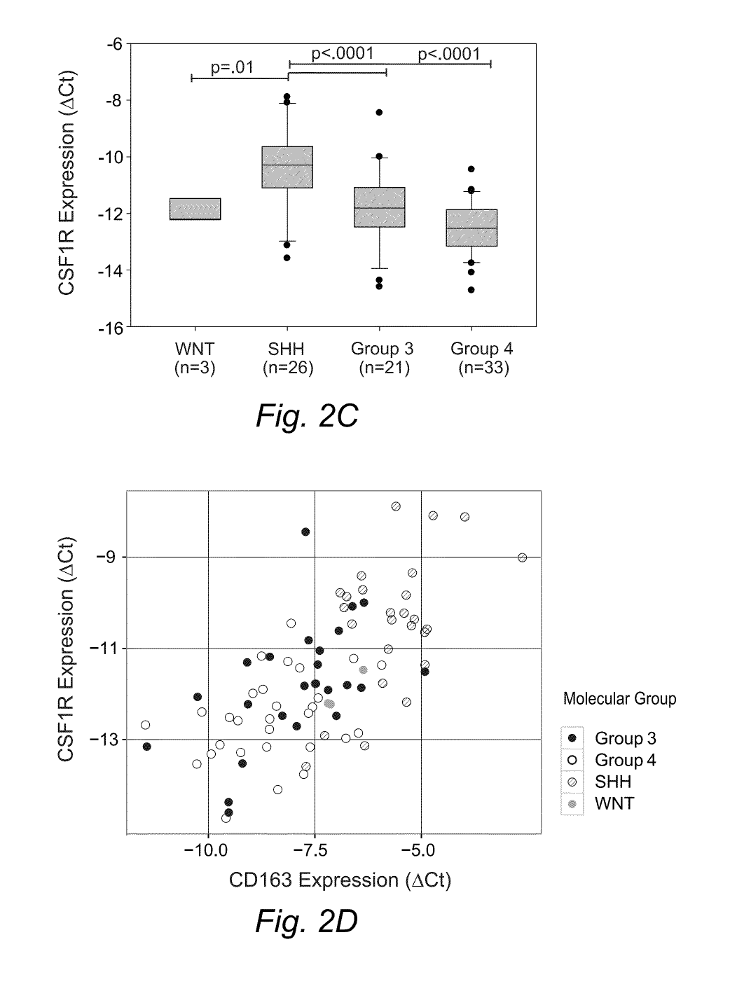

Silhouette analysis (Rousseeuw P: Silhouettes: A graphical aid to the interpretation and validation of cluster analysis. J Comput Appl Math 20:53-65, 1987) was performed to identify samples with strong membership (largest silhouette) to their assigned cluster. Samples with a silhouette width of <0.15 were deemed outliers and excluded from further analysis (FIG. 2C).

### TLDA Analysis and Gene Selection

### Gene Selection

The medulloblastoma custom TLDA card was constructed with genes related to tumor and inflammation. Tumor-related genes were selected based on previously published microarray studies in addition to our own HuEx microarray analysis. Inflammation related genes were selected based on identification of inflammation-related genes from the HuEx data. The TLDA card design used in our experiments allows for analysis of 48 genes for a given sample. Two genes (ACTB and GAPDH) were selected as housekeeping genes and used for normalization of sample data.

### Summarization and Normalization

After performing qRT-PCR reactions using the 48-gene TLDA system, all 45 genes and 2 housekeeping genes (GAPDH and ACTB) had cycle threshold (CT) values for detectible expression (CT<40) in more than 95% of specimens. The CT value of the third housekeeping gene (LDHA) was not used due to high variability. The CT values for the 45 genes were normalized to the geometric mean of the two housekeeping genes. Cycle threshold (CT) value for each gene was determined as follows: (1) Raw fluorescence values for each PCR cycle were exported from the Applied Biosystems 7900HT Version 2.3 Sequence Detection System software; (2) For each gene within each sample, a baseline value was computed as the median fluorescence from cycles 3-15. To avoid overestimating the baseline for some high-expressing genes, the upper limit of this range was adjusted to a value that was at least 3 cycles lower than the computed CT value for the gene; (3) The baseline value was subtracted from the raw fluorescence values and a LOESS smoothing function was fitted. A CT value was computed as the point where the smoothed function intersected a fixed threshold value of 0.20. The assay was considered negative if the baseline-corrected function did not intersect the fixed threshold.

### Determination of Molecular Group

A linear discriminant analysis (LDA) predictor of molecular group based on gene expression values from the custom TLDA card was performed as follows. ΔCT values of 45 genes of interest were computed as the difference between the gene CT and the geometric mean of CT values of housekeeping genes ACTB and GAPDH. Using the molecular group designation from the NMF analysis of HuEx data as the gold standard, gene-by-gene one-way ANOVA was performed, and genes that were significant in this analysis at p<0.001 were selected. In order to reduce dimensionality, principal components analysis (PCA) was performed on the selected genes, and principal components (PCs) associated with an eigenvalue >1 were retained. These PCs were all included in an LDA, again using the NMF classification as the gold standard. Leaveone-out internal cross validation (LOOCV) was performed by excluding each sample in turn, and repeating all steps above (ANOVA, gene selection, PCA, LDA), and the left out sample was classified according to the resulting LDA. The LOOCV classifications were used to compute error rates.

### Example 2

### Inflammation-Related Genes in Medulloblastoma Molecular Subgroups

We sought to identify inflammation and immunology-related genes that were differentially expressed among the molecular subgroups using HuEx gene expression data (n=168). We identified greater expression of inflammation-related genes (CD14, PTX3, CD4, CD163, CSF1R, TGFB1) in tumors of the SHH molecular subgroup compared with those of the Group 3 and Group 4 subgroups (FIG. 1C). Several of these genes have been shown to play an important role in the microenvironment of other tumors types, and in some cases have prognostic significance. (Jensen T O, et al. Macrophage markers in serum and tumor have prognostic impact in American Joint Committee on Cancer stage I/II melanoma. J Clin Oncol 2009; 27:3330-7; Locatelli M, et al. The long pentraxin PTX3 as a correlate of cancer-related inflammation and prognosis of malignancy in gliomas. J Neuroimmunol. 2013; 260:99-106). CD14, a monocytic marker present on both circulating and resident monocytes, has been show to correlate with tumor grade in gliomas (Prosniak M, et al. Glioma grade is associated with the accumulation and activity of cells bearing m2 monocyte markers. Clin Cancer Res. 2013; 19:3776-86) while TGFB1 production by glioma cells induces the infiltrating microglia/macrophages towards the M2-like phenotype.(32) CD163 is a well described marker of TAMs, and CSF1R is an important receptor which along with its ligand CSF1, controls the production, differentiation and function of TAMs (Pollard J W. Opinion: Tumour-educated macrophages promote tumour progression and metastasis. Nat Rev Cancer. Nature Publishing Group; 2004; 4:71-8; Lewis C E C, Pollard J W J. Distinct role of macrophages in different tumor microenvironments. Cancer Res. 2006; 66:605-12; Condeelis J, Pollard J W. Macrophages: obligate partners for tumor cell migration, invasion, and metastasis. Cell. 2006; 124: 263-66; Biswas S K, Mantovani A. Macrophage plasticity and interaction with lymphocyte subsets: cancer as a paradigm. Nat Immunol. 2010; 11:889-96). PTX3 is produced by macrophages that have been polarized to the M2-like phenotype via their interaction with CD4+ T-regulatory cells.(34)

Expression levels of TAM markers, CD163 and CSF1R, varied significantly among molecular subgroups (CD163 p<0.0001; CSF1R p<0.0001) and were significantly greater in tumors of the SHH subgroup compared to those in Group 3 and Group 4 (CD163, ANOVA with linear contrast p<0.0001 for both groups; CSF1R, ANOVA with linear contrast p<0.0001 for both groups). There was no statistically significant difference in CD163 or CSF1R expression between SHH tumors and WNT tumors (CD163 p=0.97; CSF1R p=0.50) (FIG. 1B and FIG. 7). These data suggest that expression of inflammation-related genes, especially those related to TAMs, can distinguish the tumor microenvironment of the SHH subgroup of medulloblastomas from Groups 3 and 4.

### Example 3

### Expression of Inflammation- and Tumor Cell-Related Genes Comprises a Molecular Subgroup Signature

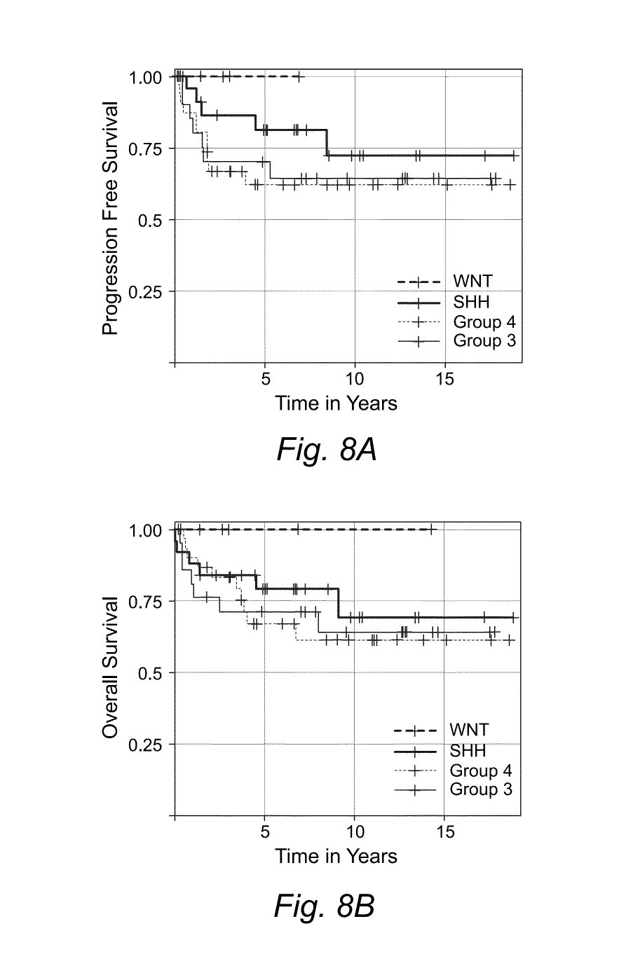

In order to identify the subgroups in a larger cohort of medulloblastoma patients and to validate expression of inflammation-related genes, we developed a robust and clinically applicable assay using the TLDA technology, a system currently being evaluated in neuroblastoma and utilized in breast cancer clinical trials (Espinosa E, et al. Comparison of prognostic gene profiles using qRT-PCR in paraffin samples: a retrospective study in patients with early breast cancer. PLoS ONE. 2009; 4:e5911; Vermeulen J, et al. Predicting outcomes for children with neuroblastoma using a multigene-expression signature: a retrospective SIOPEN/COG/GPOH study. Lancet Oncol. 2009; 10:663-71). We built a medulloblastoma-specific TLDA card containing 39 tumor-related and 6 inflammation-related genes (CD163, CSF1R, MMD, CD4, ALCAM, CXCR4) that were observed as significantly deregulated among medulloblastoma subgroups in our HuEx microarray analysis (Table 5). The TLDA gene expression profiles of medulloblastomas were then used to build and validate a 31-gene signature that could accurately predict the 4 molecular subgroups in 83 samples including two matched relapse cases (Table 2 and FIG. 2A). The estimated leave-one-out cross-validated error rate of the 31-gene signature was 2% with classification errors occurring only in samples identified as Group 4 (Tables 7-9). The patient characteristics and distribution of tumors among molecular subgroups for the 81 CHLA patients were again similar to previous published reports with 4% WNT, 31% SHH, 26% Group 3 and 39% Group 4 (Table 1). The molecular subgroups of the two relapse cases were the same as their diagnostic counterparts. All patients identified as WNT subgroup in our study cohort enjoyed long-term progression free survival, similar to previously published reports (FIG. 8).

TABLE 1
Patient characteristics by Molecular subgroup

| | WNT*† | | SHH | | Group 3 | | Group 4 | | |
|---|---|---|---|---|---|---|---|---|---|
| | No. of | | No. of | | No. of | | No. of | | |
| | Patients | % | Patients | % | Patients | % | Patients | % | P |
| Total patients | 3 | 4 | 25 | 31 | 21 | 26 | 32 | 39 | |
| Age group, years | | | | | | | | | <0.01‡ |
| ≦3 | 0 | 0 | 15 | 60 | 5 | 24 | 2 | 6 | |
| 3-6 | 0 | 0 | 5 | 20 | 6 | 29 | 11 | 34 | |
| 6-10 | 2 | 67 | 4 | 16 | 6 | 29 | 10 | 31 | |
| ≧10 | 1 | 33 | 1 | 4 | 4 | 19 | 9 | 28 | |
| Sex | | | | | | | | | 0.01‡ |
| Male | 0 | 0 | 13 | 52 | 14 | 67 | 25 | 78 | |
| Female | 3 | 100 | 12 | 48 | 7 | 33 | 7 | 22 | |
| M Stage | | | | | | | | | 0.41‡ |
| M0 | 3 | 100 | 21 | 84 | 14 | 67 | 23 | 72 | |
| M1 | 0 | 0 | 1 | 4 | 2 | 10 | 0 | 0 | |
| M2 | 0 | 0 | 0 | 0 | 1 | 5 | 0 | 0 | |
| M3 | 0 | 0 | 3 | 12 | 4 | 19 | 9 | 28 | |
| Histology | | | | | | | | | <0.001‡ |
| Desmoplastic | 0 | 0 | 13 | 52 | 1 | 5 | 2 | 6 | |
| Classic | 3 | 100 | 11 | 44 | 12 | 57 | 22 | 69 | |
| Anaplastic | 0 | 0 | 1 | 4 | 8 | 38 | 8 | 25 | |
| No. of events | 0 | 0 | 5 | 20 | 7 | 33 | 11 | 34 | 0.51§ |
| No. of deaths | 0 | 0 | 3 | 12 | 6 | 29 | 9 | 28 | 0.32§ |

*Molecular group based on TLDA analysis
†2 designated WNT based on presence of CTNNB1 mutation
‡Based on Chi-squared test
§Based on Kaplan-Meier method and log-rank test

TABLE 2
TLDA 31-gene signature

| Table 2. | | | |
|---|---|---|---|
| Gene Symbols | Gene Name | Gene Location | AUC |
| Tumor- | | | |
| related | | | |
| TERC | Telomerase RNA component | 3q26 | 0.78 |
| FOXG1 | Forkhead box G1 | 14q13 | 0.95 |
| PPP1R17 | Protein phosphatase 1, regulatory subunit 17 | 7p15 | 0.91 |
| SLC6A5 | Solute carrier family 6 (neurotransmitter transporter), member 5 | 11p15.1 | 0.82 |
| BCAT1 | Branched chain amino-acid transaminase 1, cytosolic | 12p12.1 | 0.72 |
| CBLN3 | Cerebellin 3 precursor | 14q12 | 0.81 |
| PID1 | Phosphotyrosine interaction domain containing 1 | 2q36.3 | 0.98 |
| ERG1 | Early growth response protein 1 | 5q23- | 0.63 |
| WIF1 | WNT inhibitory factor 1 | 12q14.3 | 0.51 |
| DKK2 | Dickkopf WNT signaling pathway inhibitor 2 | 4q25 | 0.71 |
| PYGL | Phosphorylase, glycogen, liver | 14q21- | 0.51 |
| TNC | Tenascin C | 9q33 | 0.82 |
| PDLIM3 | PDZ and LIM domain 3 | 4q35 | 0.89 |
| HHIP | Hedgehog interacting protein | 4q28- | 0.93 |
| SFRP1 | Secreted frizzled-related protein 1 | 8p11.21 | 0.90 |
| GLI1 | GLI family zinc finger 1 | 12q13.2- | 0.96 |
| NPR3 | Natriuretic peptide receptor C/guanylate cyclase C (atrionatriuretic | 5p14- | 0.75 |
| MYC | V-myc avian myelocytomatosis viral oncogene homolog | 8q24.21 | 0.52 |
| IMPG2 | Interphotoreceptor matrix proteoglycan 2 | 3q12.2- | 0.88 |
| GABRA5 | Gamma-aminobutyric acid (GABA) A receptor, alpha 5 | 15q12 | 0.88 |
| EOMES | Eomesodermi | 3p24.1 | 0.88 |
| MPP3 | Membrane protein, palmitoylated 3 (MAGUK p55 subfamily member | 17q21.31 | 0.95 |
| FSTL5 | Follistatin-like 5 | 4q32.3 | 0.84 |
| PDGFRA | Platelet-derived growth factor receptor, alpha polypeptide | 4q12 | 0.96 |
| OTX2 | Orthodenticle homeobox 2 | 14q22.3 | 0.96 |
| Inflammation- | | | |
| related | | | |
| CD163 | CD163 molecule | 12p13.3 | 0.94 |
| CSF1R | Colony stimulating factor 1 receptor | 5q32 | 0.78 |
| MMD | Monocyte to macrophage differentiation-associated | 17q22 | 0.97 |
| CD4 | CD4 molecule | 12p13.31 | 0.74 |
| CXCR4 | Chemokine (C-X-C motif) receptor 4 | 2q21 | 1.00 |
| ALCAM | Activated leukocyte cell adhesion molecule | 3q13.1 | 0.71 |

AUC values for distinguishing WNT and SHH from the Group 3 and 4, Four-way ANOVA p < 0.001 for all genes in signature.

TABLE 3
Coefficients of 31 signature genes

| | Molecular Subgroups | | | |
|---|---|---|---|---|
| Coefficients | Group 3 | Group 4 | SHH | WNT |
| β_{ALCAM} | −1.367776871 | −1.769739151 | −2.189418316 | 1.008437634 |
| β_{BCAT1} | −0.534636855 | 0.213866964 | 0.262004703 | −2.438541412 |
| β_{CBLN3} | −0.584693193 | −0.526325107 | 0.457666963 | −0.701506913 |
| β_{CD163} | −1.599789381 | −2.10319519 | −0.871770084 | −2.441047907 |
| β_{CD4} | −1.631846786 | −2.065302849 | −1.007242441 | −4.022360802 |
| β_{CSF1R} | −2.156824589 | −2.371630192 | −1.46330893 | −4.144571781 |
| β_{CXCR4} | −0.651280224 | −1.001717687 | 0.021241447 | −0.546590805 |
| β_{DKK2} | −0.464394152 | −0.838721573 | −1.601708055 | 2.867124319 |
| β_{EGR1} | −1.638236642 | −2.622097969 | −1.305633783 | −3.253755808 |
| β_{EOMES} | 0.073895209 | 0.379012287 | −0.234139055 | −0.583475113 |
| β_{FOXG1} | −0.359285325 | −0.330290645 | −1.289734721 | −1.304631114 |
| β_{FSTL5} | −0.833438098 | −0.95774281 | −0.573143303 | −3.281084061 |
| β_{GABRA5} | −0.356030613 | −0.835729241 | −0.760884404 | −1.228668332 |
| β_{GLI1} | −1.090841055 | −1.541170001 | −0.214207545 | −1.936838984 |
| β_{HHIP} | −0.968614578 | −1.109485984 | 0.192008331 | −2.467005014 |
| β_{IMPG2} | −0.778781414 | −1.314459562 | −2.196502209 | −0.081688486 |
| β_{MMD} | −0.450012505 | −0.471098512 | −1.69543767 | −0.79158479 |
| β_{MPP3} | −0.288985938 | 0.107019328 | −0.516841948 | −1.593216777 |
| β_{MYC} | −0.592757523 | −1.460371256 | −1.720372677 | 2.127179146 |
| β_{NPR3} | −1.027135134 | −1.473740816 | −1.092957854 | −3.806810617 |
| β_{OTX2} | 0.083120883 | 0.079927221 | −1.125548124 | 1.005084276 |
| β_{PDGFRA} | −1.242420554 | −1.575850725 | −0.653730929 | −1.136945486 |
| β_{PDLIM3} | −1.095413208 | −1.304201484 | 0.336270869 | −3.527924538 |
| β_{PID1} | −1.075829983 | −1.330710649 | −0.498279959 | −0.467160344 |
| β_{PPP1R17} | −0.680468321 | −0.425746292 | 0.314777136 | −1.143164039 |
| β_{PYGL} | −0.960527718 | −1.57236135 | −2.264456034 | 1.749578953 |
| β_{SFRP1} | −0.362708092 | −0.09727975 | 1.041796327 | −1.942569137 |
| β_{SLC6A5} | 0.236007646 | 0.541368663 | −1.134016871 | 1.745964885 |
| β_{TERC} | −0.695041239 | −0.377499193 | −1.81052649 | −0.301935345 |
| β_{TNC} | −0.593996227 | −1.059043169 | −1.41117394 | 3.022968531 |
| β_{WIF1} | −0.186942488 | −0.042140722 | −1.34875536 | 3.460480213 |
| β₀ (constant) | −108.5010223 | −157.2399902 | −142.4472809 | −228.9062195 |

TABLE 4
Patient and tumor characteristic

| | No. of Patients | % |
|---|---|---|
| Age group, years | | |
| ≦3 | 23 | 27 |
| 3-6 | 23 | 27 |
| 6-10 | 23 | 27 |
| ≧10 | 16 | 19 |
| Sex | | |
| Male | 55 | 65 |
| Female | 30 | 35 |
| M Stage | | |
| M0 | 63 | 74 |
| M1 | 3 | 4 |
| M2 | 1 | 1 |
| M3 | 18 | 21 |
| Histology | | |
| Desmoplastic | 16 | 19 |
| Classic | 52 | 61 |
| Anaplastic | 17 | 20 |
| No. of Events | 24 | 28 |
| No. Deaths | 19 | 22 |

TABLE 5
TLDA design

| Symbol | Gene Name | Gene Location | P-value | AUC |
|---|---|---|---|---|
| ALCAM | Activated leukocyte cell adhesion molecule | 3q13.1 | 0.00 | 0.71 |
| BCAT1 | Branched chain amino-acid transaminase 1, cytosolic | 12p12.1 | 0.00 | 0.72 |
| CBLN3 | Cerebellin 3 precursor | 14q12 | 0.00 | 0.81 |
| CD163 | CD163 molecule | 12p13.3 | 0.00 | 0.94 |
| CD4 | CD4 molecule | 12p13.31 | 0.00 | 0.74 |
| CHRNA6 | Cholinergic receptor, nicotinic, alpha 6 (neuronal) | 8p11.21 | 0.12 | 0.59 |
| CSF1R | Colony stimulating factor 1 receptor | 5q32 | 0.00 | 0.78 |
| CXCR4 | Chemokine (C-X-C motif) receptor 4 | 2q21 | 0.00 | 1.00 |
| DKK2 | Dickkopf WNT signaling pathway inhibitor 2 | 4q25 | 0.00 | 0.71 |
| EGR1 | Early growth response 1 | 5q31.1 | 0.00 | 0.63 |
| EOMES | Eomesodermin | 3p24.1 | 0.00 | 0.88 |
| FOXG1 | Forkhead box G1 | 14q13 | 0.00 | 0.95 |
| FSTL5 | Follistatin-like 5 | 4q32.3 | 0.00 | 0.84 |
| GABRA4 | Gamma-aminobutyric acid (GABA) A receptor, alpha 4 | 4p12 | 0.03 | 0.63 |
| GABRA5 | Gamma-aminobutyric acid (GABA) A receptor, alpha 5 | 15q12 | 0.00 | 0.88 |
| GLI1 | GLI family zinc finger 1 | 12q13.2-q13.3 | 0.00 | 0.96 |
| HDAC3 | Histone deacetylase 3 | 5q31 | 0.04 | 0.56 |
| HDAC5 | Histone deacetylase 5 | 17q21 | 0.03 | 0.72 |
| HHIP | Hedgehog interacting protein | 4q28-q32 | 0.00 | 0.93 |
| IMPG2 | Interphotoreceptor matrix proteoglycan 2 | 3q12.2-q12.3 | 0.00 | 0.88 |
| KCNA1 | Potassium voltage-gated channel, shaker-related | 12p13.32 | 0.00 | 0.65 |
| | subfamily, member 1 (episodic ataxia with myokymia) | | | |
| MMD | Monocyte to macrophage differentiation-associated | 17q22 | 0.00 | 0.97 |
| MPP3 | Membrane protein, palmitoylated 3 (MAGUK p55 | 17q21.31 | 0.00 | 0.95 |
| | subfamily member 3) | | | |
| MYC | V-myc avian myelocytomatosis viral oncogene | 8q24.21 | 0.00 | 0.52 |
| | homolog | | | |
| MYCN | V-myc avian myelocytomatosis viral oncogene | 2p24.3 | 0.02 | 0.73 |
| | neuroblastoma derived homolog | | | |
| NPR3 | Natriuretic peptide receptor C/guanylate cyclase C | 5p14-p13 | 0.00 | 0.75 |
| | (atrionatriuretic peptide receptor C) | | | |
| OAS1 | 2′-5′-oligoadenylate synthetase 1, 40/46 kDa | 12q24.2 | 0.03 | 0.77 |
| OTX2 | Orthodenticle homeobox 2 | 14q22.3 | 0.00 | 0.96 |
| PDGFRA | Platelet-derived growth factor receptor, alpha | 4q12 | 0.00 | 0.96 |
| | polypeptide | | | |
| PDLIM3 | PDZ and LIM domain 3 | 4q35 | 0.00 | 0.89 |
| PID1 | Phosphotyrosine interaction domain containing 1 | 2q36.3 | 0.00 | 0.98 |
| PPP1R17 | Protein phosphatase 1, regulatory subunit 17 | 7p15 | 0.00 | 0.91 |
| PYGL | Phosphorylase, glycogen, liver | 14q21-q22 | 0.00 | 0.51 |
| SERPINI1 | Serpin peptidase inhibitor, clade I (neuroserpin), | 3q26.1 | 0.99 | 0.53 |
| | member 1 | | | |
| SFRP1 | Secreted frizzled-related protein 1 | 8p11.21 | 0.00 | 0.90 |
| SLC6A5 | Solute carrier family 6 (neurotransmitter transporter), | 11p15.1 | 0.00 | 0.82 |
| | member 5 | | | |
| SLN | Sarcolipin | 11q22-q23 | 0.12 | 0.59 |
| SPON2 | Spondin 2, extracellular matrix protein | 4p16.3 | 0.56 | 0.51 |
| SPP1 | Secreted phosphoprotein 1 | 4q22.1 | 0.74 | 0.51 |
| STC1 | Stanniocalcin 1 | 8p21-p11.2 | 0.00 | 0.53 |
| TERC | Telomerase RNA component | 3q26 | 0.00 | 0.78 |
| TERT | Tenascin C | 9q33 | 0.01 | 0.67 |
| TGFBR2 | Transforming growth factor, beta receptor II | 3p22 | 0.15 | 0.66 |
| TNC | Tenascin C | 9q33 | 0.00 | 0.82 |
| WIF1 | WNT inhibitory factor 1 | 12q14.3 | 0.00 | 0.51 |

TABLE 6
Information for sample evaluated using HuEx. Discrepancy refers to discrepancy between previous
classification among Toronto samples using prior classification and combined classification.

| | | | | | | | Previously | Classification NMF | | |
|---|---|---|---|---|---|---|---|---|---|---|
| | | Age | | | | | Published NMF | using all available | | |
| | | Group | | | | | Classification | HuEx (Toronto & | Silhouette | |
| Sample | Gender | (Years) | Stage | Relapse | Death | Histology | (Toronto Samples) | CHLA Samples) | Width | Discrepancy |
| 529305 | F | | | | | Classic | SHH | SHH | 0.52 | No |
| 529306 | M | | | | | Classic | Group 3 | Group 3 | 0.34 | No |
| 529307 | M | | | | | Classic | SHH | SHH | 0.32 | No |
| 529308 | F | | | | | Desmoplastic | SHH | SHH | 0.53 | No |
| 529309 | M | | | | | Desmoplastic | Group 4 | Group 4 | 0.18 | No |
| 529310 | M | | | | | Classic | SHH | SHH | 0.48 | No |
| 529311 | F | | | | | Desmoplastic | SHH | SHH | 0.51 | No |
| 529312 | F | | | | | Classic | WNT | WNT | 0.26 | No |
| 529313 | F | | | | | Desmoplastic | SHH | SHH | 0.56 | No |
| 529315 | M | | | | | Anaplastic | SHH | SHH | 0.54 | No |
| 529316 | F | | | | | Classic | Group 4 | Group 4 | 0.40 | No |
| 529317 | M | | | | | Classic | Group 4 | Group 4 | 0.39 | No |
| 529318 | M | | | | | Classic | Group 3 | Group 3 | 0.01 | No |
| 529319 | M | | | | | Classic | Group 4 | Group 4 | 0.34 | No |
| 529320 | F | | | | | Desmoplastic | SHH | SHH | 0.50 | No |
| 529321 | M | | | | | Classic | Group 3 | Group 3 | 0.33 | No |
| 529322 | F | | | | | Classic | Group 4 | Group 4 | 0.43 | No |
| 529323 | M | | | | | Classic | WNT | WNT | 0.33 | No |
| 529324 | F | | | | | Classic | Group 3 | Group 3 | 0.15 | No |
| 529325 | M | | | | | Desmoplastic | SHH | SHH | 0.57 | No |
| 529326 | M | | | | | Classic | Group 4 | Group 4 | 0.24 | No |
| 529328 | F | | | | | Classic | Group 4 | Group 4 | 0.43 | No |
| 529329 | M | | | | | Classic | Group 4 | Group 4 | 0.49 | No |
| 529330 | M | | | | | Classic | Group 4 | Group 4 | 0.30 | No |
| 529331 | M | | | | | Classic | Group 4 | Group 4 | 0.26 | No |
| 529332 | F | | | | | Desmoplastic | SHH | SHH | 0.50 | No |
| 529333 | M | | | | | Classic | SHH | SHH | 0.49 | No |
| 529334 | M | | | | | Classic | Group 4 | Group 4 | 0.52 | No |
| 529335 | M | | | | | Classic | SHH | SHH | 0.48 | No |
| 529336 | F | | | | | Classic | Group 4 | Group 4 | 0.40 | No |
| 529337 | M | | | | | Classic | Group 4 | Group 4 | 0.47 | No |
| 529338 | M | | | | | Classic | WNT | WNT | 0.24 | No |
| 529339 | M | | | | | Classic | Group 3 | Group 3 | 0.32 | No |
| 529340 | M | | | | | Classic | WNT | WNT | 0.35 | No |
| 529341 | F | | | | | Classic | WNT | WNT | 0.31 | No |
| 529342 | M | | | | | Classic | Group 3 | Group 3 | 0.26 | No |
| 529343 | M | | | | | Desmoplastic | Group 3 | Group 3 | −0.01 | No |
| 529344 | M | | | | | Classic | Group 3 | Group 3 | 0.21 | No |
| 529345 | M | | | | | Classic | Group 4 | Group 4 | 0.50 | No |
| 529347 | M | | | | | Classic | Group 3 | Group 3 | 0.33 | No |
| 529349 | F | | | | | Desmoplastic | SHH | SHH | 0.58 | No |
| 529350 | F | | | | | Classic | SHH | SHH | 0.55 | No |
| 529351 | F | | | | | Classic | SHH | SHH | 0.54 | No |
| 529352 | F | | | | | Classic | SHH | SHH | 0.58 | No |
| 529353 | F | | | | | Desmoplastic | Group 4 | Group 4 | 0.42 | No |
| 529354 | M | | | | | Classic | SHH | SHH | 0.57 | No |
| 529355 | M | | | | | Desmoplastic | Group 3 | Group 3 | 0.38 | No |
| 529356 | F | | | | | Classic | SHH | SHH | 0.53 | No |
| 529357 | M | | | | | Desmoplastic | SHH | SHH | 0.47 | No |
| 529358 | M | | | | | Classic | Group 3 | Group 3 | 0.32 | No |
| 529360 | M | | | | | Classic | SHH | SHH | 0.50 | No |
| 529361 | M | | | | | Classic | Group 4 | Group 4 | 0.45 | No |
| 529362 | F | | | | | Classic | Group 3 | Group 3 | 0.18 | No |
| 529363 | M | | | | | Anaplastic | Group 4 | Group 4 | 0.33 | No |
| 529365 | M | | | | | Anaplastic | Group 4 | Group 4 | 0.24 | No |
| 529366 | F | | | | | Classic | WNT | WNT | 0.33 | No |
| 529367 | M | | | | | Anaplastic | Group 4 | Group 4 | 0.41 | No |
| 529368 | M | | | | | Classic | WNT | WNT | 0.33 | No |
| 529369 | M | | | | | Classic | Group 4 | Group 4 | 0.43 | No |
| 529370 | F | | | | | Classic | Group 3 | Group 3 | 0.31 | No |
| 529371 | M | | | | | Classic | Group 3 | Group 3 | 0.10 | No |
| 529372 | M | | | | | Classic | SHH | SHH | 0.41 | No |
| 529373 | F | | | | | Classic | SHH | SHH | 0.48 | No |
| 529374 | F | | | | | Classic | SHH | SHH | 0.55 | No |
| 529375 | M | | | | | Classic | Group 4 | Group 4 | 0.36 | No |
| 529376 | F | | | | | Classic | SHH | SHH | 0.55 | No |
| 529377 | M | | | | | Classic | Group 4 | Group 4 | 0.39 | No |
| 529378 | F | | | | | Classic | WNT | WNT | 0.27 | No |
| 529379 | M | | | | | Desmoplastic | SHH | SHH | 0.51 | No |
| 529380 | M | | | | | Classic | SHH | SHH | 0.51 | No |
| 529382 | M | | | | | Classic | Group 4 | Group 4 | 0.46 | No |
| 529383 | F | | | | | Classic | SHH | SHH | 0.45 | No |
| 529384 | M | | | | | Classic | Group 4 | Group 4 | 0.43 | No |
| 529385 | M | | | | | Classic | Group 3 | Group 3 | 0.36 | No |
| 529386 | F | | | | | Classic | Group 3 | Group 3 | 0.31 | No |
| 529387 | M | | | | | Desmoplastic | Group 3 | Group 3 | 0.08 | No |
| 529388 | M | | | | | Classic | Group 3 | Group 3 | 0.30 | No |
| 529389 | F | | | | | Classic | Group 3 | Group 3 | −0.11 | No |
| 529390 | M | | | | | Classic | Group 4 | Group 4 | 0.28 | No |
| 529391 | F | | | | | Classic | Group 4 | Group 4 | 0.43 | No |
| 529392 | M | | | | | Anaplastic | SHH | SHH | 0.23 | No |
| 529393 | M | | | | | Desmoplastic | SHH | SHH | 0.42 | No |
| 529394 | M | | | | | Unknown | Group 3 | Group 3 | 0.05 | No |
| 529395 | F | | | | | Classic | Group 3 | Group 3 | 0.08 | No |
| 529397 | F | | | | | Classic | Group 4 | Group 4 | 0.22 | No |
| 529398 | F | | | | | Classic | Group 4 | Group 4 | 0.49 | No |
| 529399 | F | | | | | Classic | Group 3 | Group 3 | 0.20 | No |
| 529400 | M | | | | | Classic | Group 3 | Group 3 | 0.17 | No |
| 529401 | M | | | | | Classic | Group 3 | Group 3 | 0.24 | No |
| 529402 | M | | | | | Classic | Group 4 | Group 4 | 0.31 | No |
| 529403 | F | | | | | Classic | Group 4 | Group 4 | 0.27 | No |
| 529404 | F | | | | | Desmoplastic | SHH | SHH | 0.58 | No |
| 529405 | M | | | | | Classic | SHH | SHH | 0.36 | No |
| 529406 | M | | | | | Anaplastic | Group 3 | Group 3 | 0.10 | No |
| 529407 | M | | | | | Classic | Group 4 | Group 4 | 0.38 | No |
| 529314 | M | | | | | Classic | Group 4 | Group 3 | −0.12 | Yes |
| 529327 | F | | | | | Anaplastic | SHH | WNT | −0.05 | Yes |
| 529346 | F | | | | | Anaplastic | Group 3 | WNT | −0.07 | Yes |
| 529348 | M | | | | | Desmoplastic | Group 4 | Group 3 | −0.18 | Yes |
| 529359 | F | | | | | Classic | Group 4 | Group 3 | −0.05 | Yes |
| 529364 | M | | | | | Classic | Group 3 | Group 4 | 0.22 | Yes |
| 529381 | F | | | | | Classic | SHH | WNT | 0.03 | Yes |
| 529396 | M | | | | | Classic | Group 4 | Group 3 | −0.02 | Yes |
| S1071 | M | <3 | M0 | No | No | Desmoplastic | | SHH | 0.54 | N/A |
| S2025 | M | <3 | M0 | No | No | Desmoplastic | | SHH | 0.51 | N/A |
| S2046 | M | 6-10 | M0 | No | No | Classic | | Group 4 | 0.37 | N/A |
| S2053 | F | 6-10 | M0 | Yes | Yes | Classic | | SHH | 0.39 | N/A |
| S2056 | M | <3 | M0 | No | No | Classic | | SHH | 0.52 | N/A |
| S4018 | M | 6-10 | M0 | Yes | Yes | Classic | | Group 4 | 0.23 | N/A |
| S4021 | M | 6-10 | M0 | Yes | Yes | Desmoplastic | | Group 4 | 0.21 | N/A |
| S4022 | M | <3 | M0 | Yes | Yes | Classic | | Group 3 | 0.37 | N/A |
| S4025 | M | 6-10 | M3 | Yes | Yes | Classic | | Group 4 | 0.52 | N/A |
| S4029 | F | <3 | M0 | Yes | Yes | Desmoplastic | | SHH | 0.35 | N/A |
| S4030 | F | <3 | M3 | No | No | Desmoplastic | | SHH | 0.51 | N/A |
| S4032 | M | >10 | M0 | Yes | Yes | Anaplastic | | SHH | 0.47 | N/A |
| S4033 | M | <3 | M0 | Yes | Yes | Classic | | Group 4 | 0.20 | N/A |
| S4036 | F | 3-6 | M0 | No | No | Classic | | SHH | 0.49 | N/A |
| S4037 | F | 6-10 | M0 | No | No | Desmoplastic | | SHH | 0.49 | N/A |
| S4038 | M | <3 | M0 | No | No | Classic | | Group 3 | 0.33 | N/A |
| S4041 | M | >10 | M3 | No | No | Classic | | Group 4 | 0.26 | N/A |
| S4042 | M | <3 | M0 | No | No | Desmoplastic | | SHH | 0.44 | N/A |
| S4044 | F | >10 | M0 | No | No | Classic | | WNT | 0.36 | N/A |
| S4045 | M | 3-6 | M0 | No | No | Desmoplastic | | SHH | 0.28 | N/A |
| S4046 | M | >10 | M0 | No | No | Classic | | Group 4 | 0.33 | N/A |
| S4047 | M | 3-6 | M0 | No | No | Classic | | Group 4 | 0.26 | N/A |
| S4050 | M | <3 | M0 | No | No | Classic | | SHH | 0.53 | N/A |
| S4051 | M | 6-10 | M0 | No | No | Classic | | SHH | 0.39 | N/A |
| S4052 | F | <3 | M0 | No | No | Desmoplastic | | SHH | 0.43 | N/A |
| S4053 | M | >10 | M0 | No | No | Classic | | Group 4 | 0.46 | N/A |
| S4054 | M | 3-6 | M0 | No | No | Classic | | Group 4 | 0.40 | N/A |
| S4055 | M | 3-6 | M0 | No | No | Classic | | Group 4 | 0.28 | N/A |
| S4057 | M | >10 | M0 | No | No | Anaplastic | | Group 3 | 0.18 | N/A |
| S4061 | F | 6-10 | M3 | No | No | Anaplastic | | Group 3 | 0.23 | N/A |
| S4073 | M | >10 | M0 | No | No | Classic | | Group 4 | 0.22 | N/A |
| S4103 | M | 6-10 | M0 | No | No | Classic | | Group 4 | 0.45 | N/A |
| S4108 | F | <3 | M0 | No | No | Classic | | SHH | 0.56 | N/A |
| S4111 | F | 3-6 | M0 | No | No | Classic | | Group 3 | 0.21 | N/A |
| S4112 | M | 3-6 | M3 | No | No | Anaplastic | | Group 3 | 0.21 | N/A |
| S4114 | M | 3-6 | M0 | No | No | Desmoplastic | | SHH | 0.54 | N/A |
| S4115 | M | 3-6 | M3 | No | No | Desmoplastic | | SHH | 0.51 | N/A |
| S4117 | F | >10 | M3 | No | No | Classic | | Group 4 | 0.45 | N/A |
| S4118 | M | <3 | M0 | Yes | No | Classic | | Group 4 | 0.49 | N/A |
| S4120 | F | 6-10 | M0 | No | No | Anaplastic | | Group 3 | 0.30 | N/A |
| S4122 | F | <3 | M0 | No | No | Desmoplastic | | SHH | 0.41 | N/A |
| S4123 | F | 6-10 | M0 | No | No | Classic | | Group 4 | 0.26 | N/A |
| S4124 | M | <3 | M1 | No | No | Classic | | SHH | 0.54 | N/A |
| S4125 | M | >10 | M0 | No | No | Desmoplastic | | Group 4 | 0.21 | N/A |
| S4126 | F | 3-6 | M0 | Yes | No | Classic | | SHH | 0.27 | N/A |
| S4128 | F | <3 | M3 | No | No | Desmoplastic | | SHH | 0.40 | N/A |
| S4129 | M | >10 | M3 | No | No | Classic | | Group 4 | 0.25 | N/A |
| S1050 | M | 6-10 | M0 | Yes | Yes | Classic | | Group 3 | 0.10 | N/A |
| S1076 | F | 3-6 | M0 | No | No | Classic | | Group 3 | −0.08 | N/A |
| S1080 | M | 6-10 | M0 | No | No | Classic | | Group 3 | 0.10 | N/A |
| S2013 | M | >10 | M3 | Yes | Yes | Classic | | Group 4 | 0.46 | N/A |
| S2065 | M | 6-10 | M0 | No | No | Classic | | WNT | −0.01 | N/A |
| S4017 | M | <3 | M3 | No | No | Classic | | SHH | 0.47 | N/A |
| S4020 | M | 3-6 | M3 | Yes | Yes | Anaplastic | | Group 3 | 0.09 | N/A |
| S4023 | M | <3 | M2 | Yes | Yes | Anaplastic | | WNT | −0.18 | N/A |
| S4035 | M | 6-10 | M1 | Yes | Yes | Classic | | Group 3 | 0.09 | N/A |
| S4039 | F | 3-6 | M2 | Yes | Yes | Classic | | WNT | 0.02 | N/A |
| S4056 | F | 6-10 | M3 | Yes | No | Classic | | Group 3 | −0.08 | N/A |
| S4058 | M | 6-10 | M0 | No | No | Classic | | Group 3 | 0.11 | N/A |
| S4063 | M | 3-6 | M0 | No | No | Classic | | Group 3 | 0.13 | N/A |
| S4105 | M | <3 | M0 | No | No | Classic | | WNT | 0.04 | N/A |
| S4106 | M | <3 | M0 | No | No | Anaplastic | | WNT | 0.12 | N/A |
| S4110 | M | 3-6 | M0 | No | No | Anaplastic | | Group 3 | −0.02 | N/A |
| S4121 | M | >10 | M0 | No | No | Classic | | Group 3 | 0.03 | N/A |
| S4127 | M | 3-6 | M0 | No | No | Anaplastic | | WNT | −0.13 | N/A |

N/A: not applicable.

TABLE 7
TLDA 31-gene molecular classification of core samples. Core Samples 1: Core samples
HuEx molecular group classification. Core Samples2: Core samples molecular group
classification by TLDA 31-gene (Leave-one-out cross validation). Core Samples3:
Core samples molecular group classification by TLDA 31-gene (Resubstitution).

| | | | | | | | Core | Core | Core | | |
|---|---|---|---|---|---|---|---|---|---|---|---|
| Sample | Sex | Age | Stage | Relapse | Death | Histology | Samples¹ * | Samples² | Samples³ | Discrepancy | |
| S1037 | F | 6-10 | M0 | No | No | Classic | WNT | WNT | WNT | No | ** |
| S1071 | M | <3 | M0 | No | No | Desmoplastic | SHH | SHH | SHH | No | |
| S2025 | M | <3 | M0 | No | No | Desmoplastic | SHH | SHH | SHH | No | |
| S2034 | F | 6-10 | M0 | No | No | Classic | WNT | WNT | WNT | No | ** |
| S2046 | M | 6-10 | M0 | No | No | Classic | Group 4 | Group 4 | Group 4 | No | |
| S2053 | F | 6-10 | M0 | Yes | Yes | Classic | SHH | SHH | SHH | No | |
| S2056 | M | <3 | M0 | No | No | Classic | SHH | SHH | SHH | No | |
| S4018 | M | 6-10 | M0 | Yes | Yes | Classic | Group 4 | Group 4 | Group 4 | No | |
| S4021 | M | 6-10 | M0 | Yes | Yes | Desmoplastic | Group 4 | Group 4 | Group 4 | No | |
| S4022 | M | <3 | M0 | Yes | Yes | Classic | Group 3 | Group 3 | Group 3 | No | |
| S4025 | M | 6-10 | M3 | Yes | Yes | Classic | Group 4 | Group 4 | Group 4 | No | |
| S4029 | F | <3 | M0 | Yes | Yes | Desmoplastic | SHH | SHH | SHH | No | |
| S4030 | F | <3 | M3 | No | No | Desmoplastic | SHH | SHH | SHH | No | |
| S4032 | M | >10 | M0 | Yes | Yes | Anaplastic | SHH | SHH | SHH | No | |
| S4033 | M | <3 | M0 | Yes | Yes | Classic | Group 4 | Group 4 | Group 4 | No | |
| S4036 | F | 3-6 | M0 | No | No | Classic | SHH | SHH | SHH | No | |
| S4037 | F | 6-10 | M0 | No | No | Desmoplastic | SHH | SHH | SHH | No | |
| S4038 | M | <3 | M0 | No | No | Classic | Group 3 | Group 3 | Group 3 | No | |
| S4041 | M | >10 | M3 | No | No | Classic | Group 4 | Group 4 | Group 4 | No | |
| S4042 | M | <3 | M0 | No | No | Desmoplastic | SHH | SHH | SHH | No | |
| S4044 | F | >10 | M0 | No | No | Classic | WNT | WNT | WNT | No | |
| S4045 | M | 3-6 | M0 | No | No | Desmoplastic | SHH | SHH | SHH | No | |
| S4046 | M | >10 | M0 | No | No | Classic | Group 4 | Group 4 | Group 4 | No | |
| S4047 | M | 3-6 | M0 | No | No | Classic | Group 4 | Group 4 | Group 4 | No | |
| S4050 | M | <3 | M0 | No | No | Classic | SHH | SHH | SHH | No | |
| S4051 | M | 6-10 | M0 | No | No | Classic | SHH | SHH | SHH | No | |
| S4052 | F | <3 | M0 | No | No | Desmoplastic | SHH | SHH | SHH | No | |
| S4053 | M | >10 | M0 | No | No | Classic | Group 4 | Group 4 | Group 4 | No | |
| S4054 | M | 3-6 | M0 | No | No | Classic | Group 4 | Group 4 | Group 4 | No | |
| S4055 | M | 3-6 | M0 | No | No | Classic | Group 4 | Group 4 | Group 4 | No | |
| S4057 | M | >10 | M0 | No | No | Anaplastic | Group 3 | Group 3 | Group 4 | Yes | |
| S4061 | F | 6-10 | M3 | No | No | Anaplastic | Group 3 | Group 3 | Group 3 | No | |
| S4073 | M | >10 | M0 | No | No | Classic | Group 4 | Group 4 | Group 4 | No | |
| S4103 | M | 6-10 | M0 | No | No | Classic | Group 4 | Group 4 | Group 4 | No | |
| S4108 | F | <3 | M0 | No | No | Classic | SHH | SHH | SHH | No | |
| S4111 | F | 3-6 | M0 | No | No | Classic | Group 3 | Group 3 | Group 3 | No | |
| S4112 | M | 3-6 | M3 | No | No | Anaplastic | Group 3 | Group 3 | Group 3 | No | |
| S4114 | M | 3-6 | M0 | No | No | Desmoplastic | SHH | SHH | SHH | No | |
| S4115 | M | 3-6 | M3 | No | No | Desmoplastic | SHH | SHH | SHH | No | |
| S4117 | F | >10 | M3 | No | No | Classic | Group 4 | Group 4 | Group 4 | No | |
| S4118 | M | <3 | M0 | Yes | No | Classic | Group 4 | Group 4 | Group 4 | No | |
| S4120 | F | 6-10 | M0 | No | No | Anaplastic | Group 3 | Group 3 | Group 3 | No | |
| S4122 | F | <3 | M0 | No | No | Desmoplastic | SHH | SHH | SHH | No | |
| S4123 | F | 6-10 | M0 | No | No | Classic | Group 4 | Group 4 | Group 4 | No | |
| S4124 | M | <3 | M1 | No | No | Classic | SHH | SHH | SHH | No | |
| S4125 | M | >10 | M0 | No | No | Desmoplastic | Group 4 | Group 4 | Group 4 | No | |
| S4126 | F | 3-6 | M0 | Yes | No | Classic | SHH | SHH | SHH | No | |
| S4128 | F | <3 | M3 | No | No | Desmoplastic | SHH | SHH | SHH | No | |
| S4129 | M | >10 | M3 | No | No | Classic | Group 4 | Group 4 | Group 4 | No | |

* Molecular subgroups of core samples used to build TLDA classification are derived from HuEx data where sample's silhotte >0.15 or those with known Beta-Catenin mutation. Discrepancy: discrepancy TLDA 31-gene and HuEx classifications.
** Known beta catenin mutation. Age: age group in years.

TABLE 8
TLDA 31-gene classification for samples without HuEx data

| | | Age | | | | | Mol Group by |
|---|---|---|---|---|---|---|---|
| | | Group | | | | | TLDA 31-gene |
| Sample | Gender | (Years) | Stage | Relapse | Death | Histology | signature |
| S4106 | M | <3 | M0 | No | No | Anaplastic | Group 3 |
| S4058 | M | 6-10 | M0 | No | No | Classic | Group 3 |
| S1080 | M | 6-10 | M0 | No | No | Classic | Group 3 |
| S1050 | M | 6-10 | M0 | Yes | Yes | Classic | Group 3 |
| S4020 | M | 3-6 | M3 | Yes | Yes | Anaplastic | Group 4 |
| S4035 | M | 6-10 | M1 | Yes | Yes | Classic | Group 3 |
| S4105 | M | <3 | M0 | No | No | Classic | SHH |
| S4121 | M | >10 | M0 | No | No | Classic | Group 3 |
| S2065 | M | 6-10 | M0 | No | No | Classic | SHH |
| S4110 | M | 3-6 | M0 | No | No | Anaplastic | Group 4 |
| S1076 | F | 3-6 | M0 | No | No | Classic | Group 4 |
| S4056 | F | 6-10 | M3 | Yes | No | Classic | Group 4 |
| S4127 | M | 3-6 | M0 | No | No | Anaplastic | Group 4 |
| S4023 | M | <3 | M2 | Yes | Yes | Anaplastic | Group 3 |
| S1018 | M | 6-10 | M3 | Yes | Yes | Anaplastic | Group 4 |
| S1029 | M | 3-6 | M0 | Yes | Yes | Classic | Group 4 |
| S1030 | F | <3 | M0 | Yes | No | Desmoplastic | SHH |
| S1030* | F | <3 | M0 | Yes | No | Classic | SHH |
| S1032 | F | >10 | M0 | No | No | Anaplastic | Group 4 |
| S1039 | M | >10 | M0 | No | No | Classic | Group 4 |
| S4024 | F | 6-10 | M3 | Yes | Yes | Anaplastic | Group 4 |
| S4027 | F | 3-6 | M3 | Yes | Yes | Classic | Group 3 |
| S4028 | F | 3-6 | M0 | Yes | Yes | Anaplastic | Group 4 |
| S4033* | M | <3 | M0 | No | No | Classic | Group 4 |
| S4040 | M | <3 | M1 | Yes | Yes | Anaplastic | Group 3 |
| S4048 | F | 3-6 | M3 | No | No | Classic | Group 3 |
| S4059 | F | >10 | M0 | No | No | Classic | Group 3 |
| S4062 | M | 3-6 | M0 | No | No | Classic | Group 4 |
| S4095 | F | <3 | M0 | No | No | Classic | SHH |
| S4104 | F | >10 | M0 | No | No | Anaplastic | Group 3 |
| S4107 | M | 3-6 | M0 | No | No | Desmoplastic | Group 3 |
| S4116 | M | 3-6 | M0 | Yes | No | Classic | Group 3 |
| S4133 | M | 3-6 | M3 | No | No | Classic | Group 4 |
| S4184 | M | 6-10 | M0 | No | No | Anaplastic | Group 4 |

*Relapse sample

TABLE 9
TLDA 31-gene confusion matrix

| | | HuEx Classification | | | | |
|---|---|---|---|---|---|---|
| | | WNT | SHH | Group 3 | Group 4 | Total |
| TLDA | WNT | 3 | | | | 3 |
| LOOCV | SHH | | 26 | | | 26 |
| Classification | Group 3 | | | 20 | 1 | 21 |
| | Group 4 | | | | 33 | 33 |
| | Total | 3 | 26 | 20 | 34 | 83 |

Among the 31 genes included in our signature, increased expression of WIF1, DKK2, PGYL, and TNC was associated with WNT tumors, HHIP, PDLIM3, SFRP1 and GLI1 associated with SHH tumors, MYC, IMPG2, NPR3 associated with Group 3, and KCNA, MPP3, and EOMES associated with Group 4 tumors. These data are consistent with previous microarray-based publications indicating differential expression of these genes among molecular groups of medulloblastomas (Kool M, et al. Integrated genomics identifies five medulloblastoma subtypes with distinct genetic profiles, pathway signatures and clinicopathological features. PLoS ONE. 2008; 3:e3088; Northcott P A, et al. Medulloblastoma Comprises Four Distinct Molecular Variants. J Clin Oncol. 2011; 29:1408-14; Cho Y-J, et al. Integrative genomic analysis of medulloblastoma identifies a molecular subgroup that drives poor clinical outcome. J Clin Oncol. 2011; 29:1424-30; Schwalbe E C, et al. Rapid diagnosis of medulloblastoma molecular subgroups. Clin Cancer Res. 2011; 17:1883-94; Kool M, et al. Molecular subgroups of medulloblastoma: an international meta-analysis of transcriptome, genetic aberrations, and clinical data of WNT, SHH, Group 3, and Group 4 medulloblastomas. Acta Neuropathol. Springer-Verlag; 2012; 123:473-84).

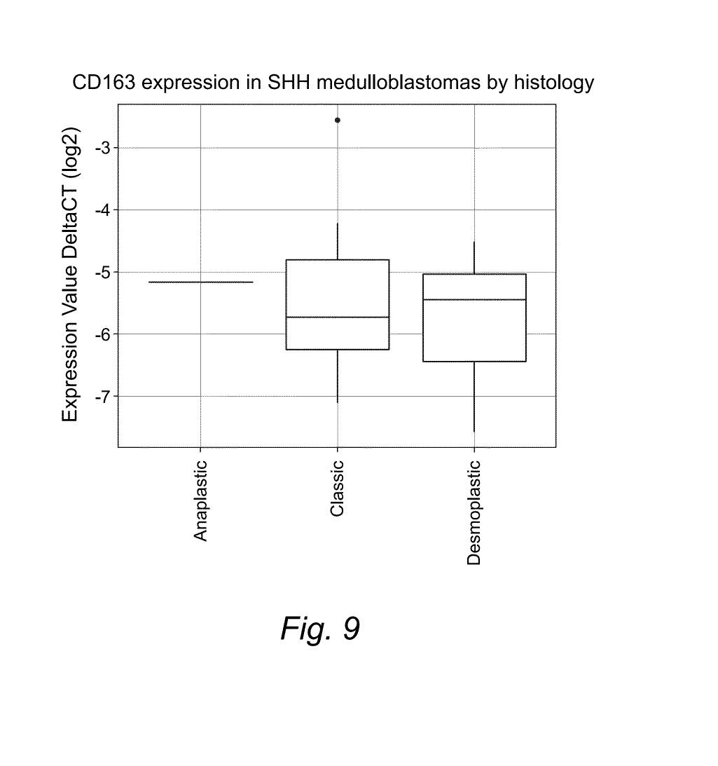

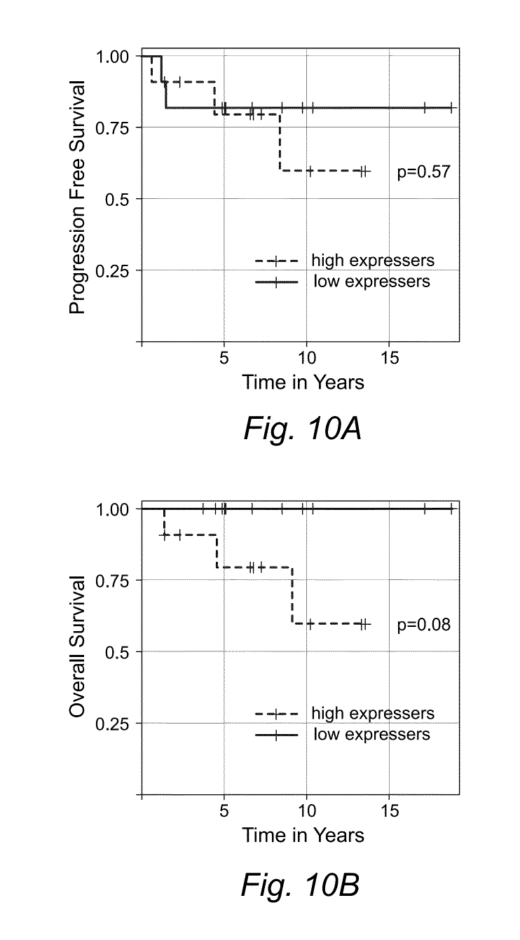

We also identified several novel genes, which were differentially expressed among medulloblastoma molecular subgroups and contributed to their accurate identification (Table 2). Notably, the TAM-associated genes CD163 and CSF1R were differentially expressed among molecular subgroups with increased expression in tumors of the SHH subgroup compared to those in Groups 3 and 4 (CD163 p<0.0001; CSF1R p<0.0001 for all pairwise comparisons) and contributed to the 31-gene signature predictive of molecular subgroups (FIGS. 2B and 2C). A gene-gene correlation was observed between CD163 and CSF1R (Spearman r=0.67, FIG. 2D) suggestive of co-expression of these two genes most likely by TAMs. There was no difference in CD163 expression in SHH tumors with desmoplastic histology compared to those with classic histology (FIG. 9). The median gene expression of CD163 among the 22 patients with SHH medulloblastoma was used to define low- and high-CD163 expressers. Ten-year overall survival (OS) for patients in these two groups was different but did not reach statistical significance (FIG. 10; CD163 high- vs. low-expresser, 58% vs. 100% respectively, p=0.08).

### Example 4

### Pattern of Infiltration of Macrophages in SHH Medulloblastoma

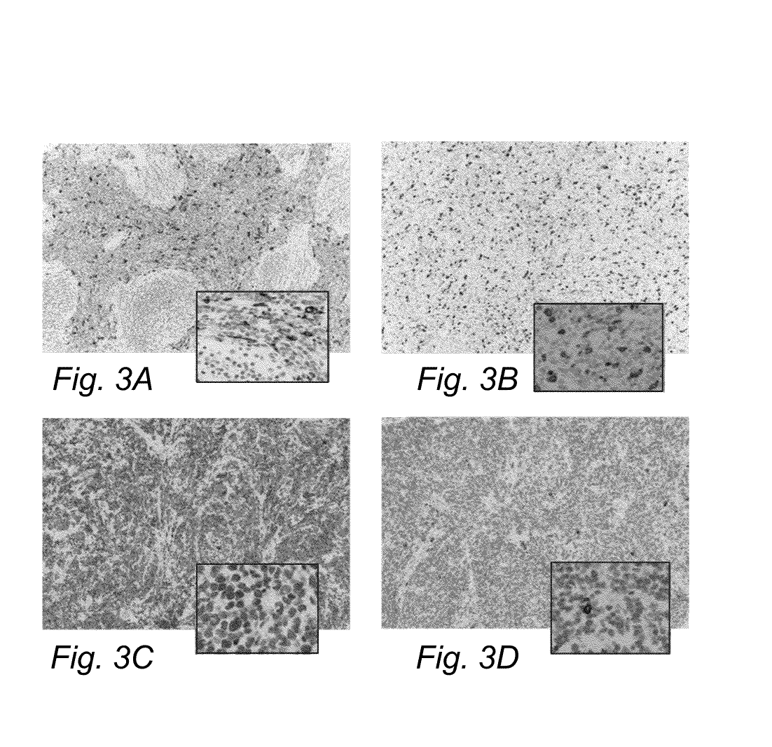

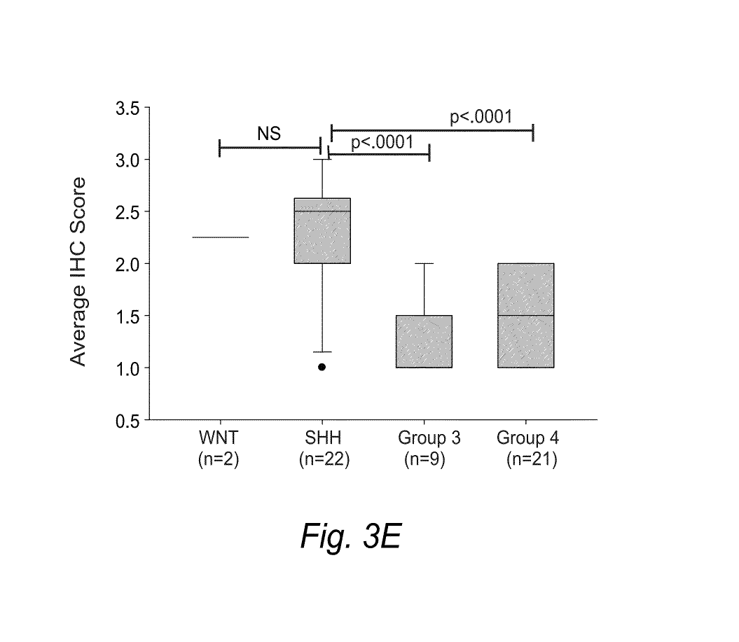

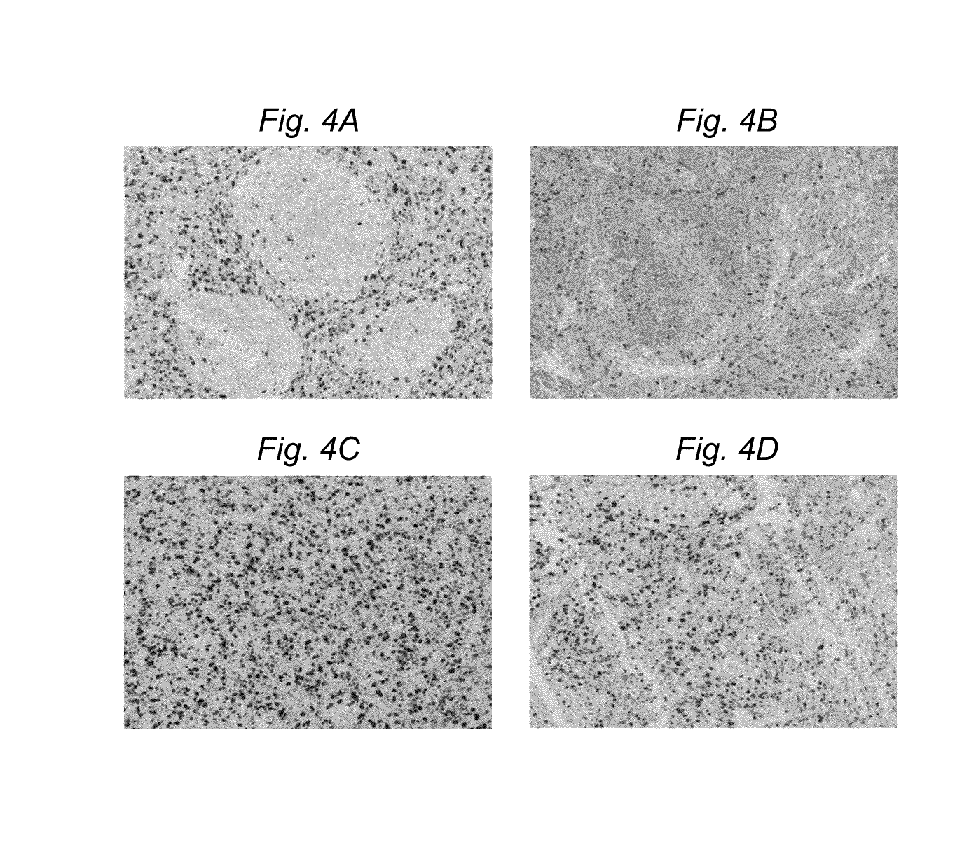

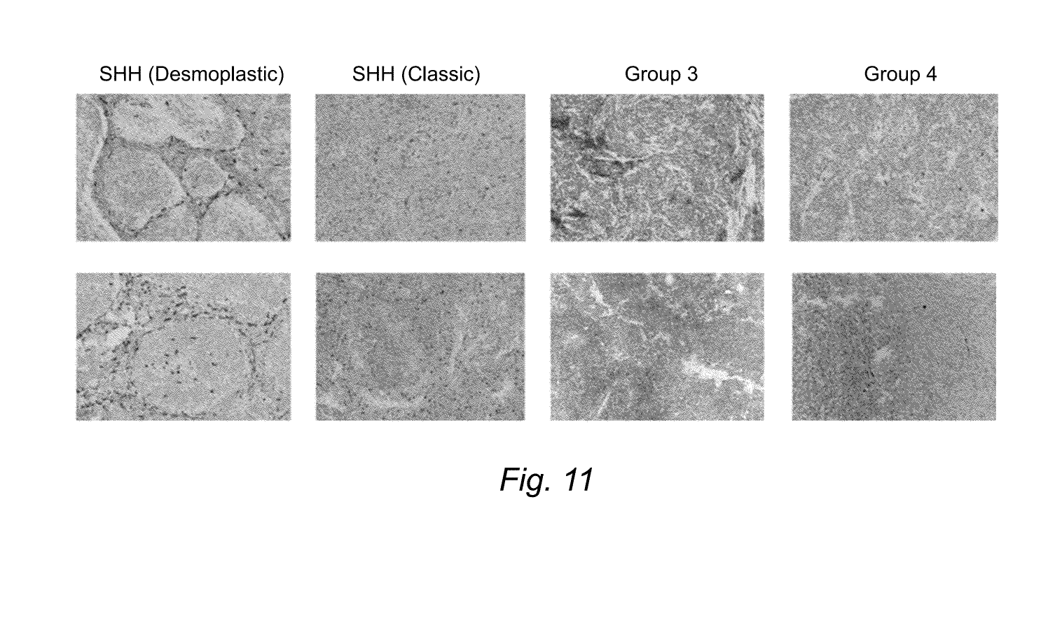

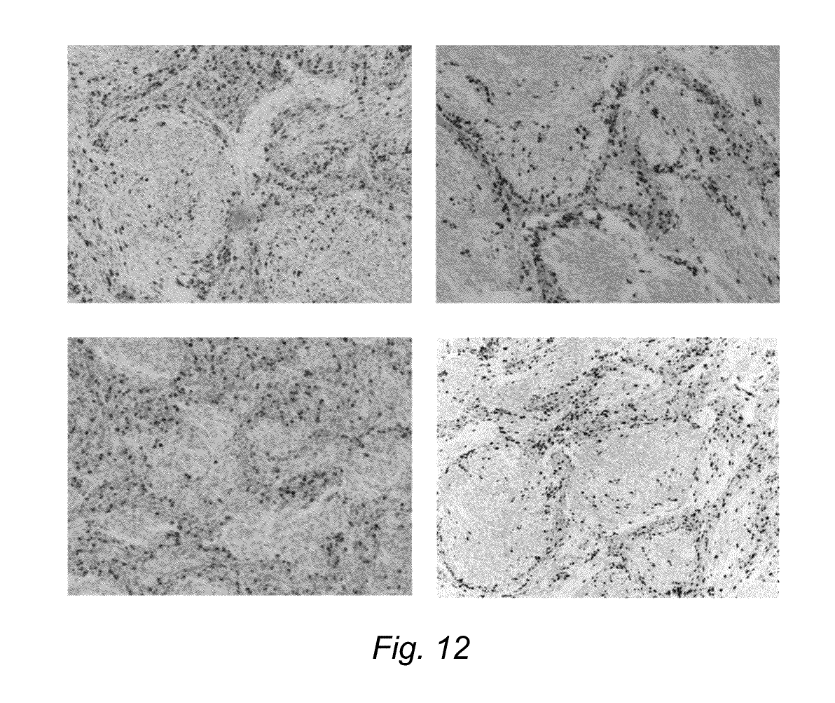

We next performed IHC analysis of 54 paraffin-embedded medulloblastoma tumors using antibodies directed against CD163 to assess the extent and pattern of TAM infiltration in medulloblastomas. There was a significant difference in macrophage infiltration among molecular subgroups (p<0.0001) with significantly greater numbers of macrophages observed in tumors of the SHH subgroup compared to those in Group 3 and Group 4 (p<0.0001 for both comparisons) (FIG. 3). There was no statistically significant difference in the number of intra-tumoral CD163+ macrophages between SHH tumors and WNT tumors, however there were only two WNT samples available for evaluation. Among tumors with an IHC score >2.5 (n=16), 94% were SHH tumors and the remaining 6% were WNT tumors. The SHH tumors with desmoplastic histology exhibited a distinct pattern of macrophage infiltration in the inter-nodular, poorly differentiated areas while sparing the more differentiated nodules (FIG. 3 and FIG. 11). Interestingly in the subset of SHH medulloblastomas with classic histology, CD163+ macrophages sometimes loosely recapitulated this lobular organization. The areas of macrophage infiltration in the SHH tumors also corresponded with areas of proliferating cells as evidenced by positive staining for Ki-67, a nuclear marker of cell proliferation, which was performed in subset of 23 tumor samples (FIG. 4 and FIG. 12).

Treatment strategies aimed at improving survival of young children with medulloblastoma by avoiding radiation therapy and its neurocognitive sequelae require identification of novel subgroup-specific targets. Our study suggests for the first time that TAMs contribute to the microenvironment of a childhood brain tumor and demonstrates their prevalence in tumors of children with SHH subgroup of medulloblastoma. We show that expression of inflammation-related genes including TAM-related genes, CD163 and CSF1R, is higher in SHH as compared to the other medulloblastoma subgroups. A 31-gene signature, inclusive of both inflammatory and tumor cell genes, enables proper identification of molecular subgroups with 98% accuracy. The 31-gene expression scoring model has clinical applicability and could be of use for risk-stratification, while identification of TAMs in SHH tumors uncovers a previously unrecognized potential target for therapy.

While targeted therapy with SHH pathway inhibitors shows tremendous promise, it has become clear that novel treatments that overcome mechanisms of resistance to these inhibitors need to be identified to improve overall survival (Rudin C M, et al. Treatment of medulloblastoma with hedgehog pathway inhibitor GDC-0449. N Engl J Med. 2009; 361:1173-78). In recent years, the concept of inflammatory cells in the tumor microenvironment as critical participants in tumor progression has gained acceptance. Large numbers of infiltrating TAMs are predictive of a poor prognosis in many adult cancers (Steidl C, et al. Tumor-associated macrophages and survival in classic Hodgkin's lymphoma. N Engl J Med. 2010; 362:875-85; Zhang B C, et al. Tumor-associated macrophages infiltration is associated with peritumoral lymphangiogenesis and poor prognosis in lung adenocarcinoma. Med Oncol. 2011; 28:1447-52; DeNardo D G, et al. Leukocyte complexity predicts breast cancer survival and functionally regulates response to chemotherapy. Cancer Discov. 2011; 1:54-67), and a 14-gene signature inclusive of 5 genes representing TAMs has been shown to predict progression-free survival in patients with metastatic neuroblastoma (Asgharzadeh S, et al. Clinical significance of tumor-associated inflammatory cells in metastatic neuroblastoma J Clin Oncol. 2012; 30:3525-32). Our study also suggests a prognostic role for expression of CD163 among patients with SHH medulloblastoma but validation with larger number of samples is needed. The tumor microenvironment also plays an important role in drug resistance mechanisms of tumors. Co-culture of leukemia cells with stromal cells allows for environment-mediated drug resistance (EMDR) to tyrosine kinase inhibitors (Feldhahn N, et al. Environment-mediated drug resistance in Bcr/Abl-positive acute lymphoblastic leukemia. Oncoimmunology. 2012; 1:618-29). This EMDR is associated with differential regulation of inflammation-related genes. TAMs produce cytokines which activate STAT3 and Hedgehog signals in colon and lung cancer stem cells rendering them resistant to chemotherapy (Jinushi M, et al. Tumor-associated macrophages regulate tumorigenicity and anticancer drug responses of cancer stem/initiating cells. Proc Natl Acad Sci USA. 2011; 108:12425-30). This suggests that combination therapy aimed at targeting the microenvironment in addition to the tumors cells may improve the response to chemotherapy and decrease the risk of development of EMDR.

In this study, we show that the expression of inflammation-related genes, especially macrophage markers CD163 and CSF1R, are highest in the SHH subgroup of medulloblastomas, which was validated by the IHC analyses. The pro-tumor effects of TAMs on tumor pathogenesis have been shown in de novo epithelial carcinogenesis in mice through production of cytokines such as IL6, IL10, and IL4 that stimulate tumor growth and angiogenesis (Coussens L M, Werb Z. Inflammation and cancer. Nature. Nature Publishing Group; 2002; 420:860-7). Co-culture of neuroblastoma cells with peripheral blood monocytes or mesenchymal cells also increases tumor cell proliferation through IL-6 and STAT3 dependent mechanisms. In a prostate cancer model, macrophages induce CCL4 production which promotes tumorigenesis through STAT3 activation (Fang L-Y, et al. Infiltrating Macrophages Promote Prostate Tumorigenesis via Modulating Androgen Receptor-Mediated CCL4-STAT3 Signaling. Cancer Res. 2013; 73:5633-46), while glioblastoma conditioned media protects TAM survival demonstrating the cross-talk between tumor cells and TAMs. The TAMs in SHH medulloblastoma are located near the proliferating tumor cells, as identified by Ki-67 marker, and points to their likely role in creating a pro-growth tumor microenvironment.

Infiltration of TAMs into the tumor microenvironment can be reduced by CSF1R inhibition (Hume D A, MacDonald K P A. Therapeutic applications of macrophage colony-stimulating factor-1 (CSF-1) and antagonists of CSF-1 receptor (CSF-1R) signaling. Blood. 2012; 119:1810-20). In a transgenic murine model of mammary adenocarcinoma, blockade of CSF1R signaling leads to a decrease in intra-tumoral TAMs resulting in increased sensitivity to chemotherapy. Administration of a CSF1R antagonist in combination with paclitaxel leads to a decrease in primary tumor progression as well as decreased rates of pulmonary metastasis and overall survival when compared to mice treated with paclitaxel alone (DeNardo D G, et al. Leukocyte complexity predicts breast cancer survival and functionally regulates response to chemotherapy. Cancer Discov. 2011; 1:54-67) Inhibition of CSF1R in several pre-clinical models of pro-neural glioblastoma multiforme repolarizes TAMs away from the M2 phenotype resulting in a subsequent decrease in tumorigenesis (Pyonteck S M, et al. CSF-1R inhibition alters macrophage polarization and blocks glioma progression. Nat Med. 2013; 19:1264-72). Our finding of TAMs with high expression of CSF1R in SHH medulloblastomas provides a novel therapeutic target. Radiation-avoiding treatment strategies could be combined with therapies aimed at blocking pathways mediating macrophage recruitment, polarization, and cross-talk with tumor cells.

Our current study also defines a clinically applicable 31-gene expression signature that identifies the four molecular subgroups of medulloblastomas with a 2% misclassification rate. Our group and others have demonstrated the clinical utility of these TLDA assays in childhood and adult clinical trials (Espinosa E, et al. Comparison of prognostic gene profiles using qRT-PCR in paraffin samples: a retrospective study in patients with early breast cancer. PLoS ONE. 2009; 4:e5911; Vermeulen J, et al. Predicting outcomes for children with neuroblastoma using a multigene-expression signature: a retrospective SIOPEN/COG/GPOH study. Lancet Oncol. 2009; 10:663-71). While current techniques such as IHC, fluorescent in situ hybridization (FISH), and cytogenetics could be used to identify only a portion of medulloblastomas subgroups (Ellison D W, et al. Definition of Disease-Risk Stratification Groups in Childhood Medulloblastoma Using Combined Clinical, Pathologic, and Molecular Variables. J Clin Oncol. 2011; 29:1400-7; Schwalbe E C, et al. Rapid diagnosis of medulloblastoma molecular subgroups. Clin Cancer Res. 2011; 17:1883-94), our proposed 31-gene signature provides a rapid and highly accurate assay for determining these subgroups. Implementation of this assay could also be used in protocols aimed at avoiding or delaying radiation therapy in children with WNT or SHH tumors, or identification of children with Group 3 of Group 4 tumors for novel therapies. As molecular subgroup becomes part of risk stratification, it will be important to have tools adept at making that determination.

In summary, our study reports the first evidence of the presence of TAMs in pediatric medulloblastoma and provides a 31-gene signature, inclusive of macrophage-associated genes, that accurately determines medulloblastoma subgroups. The increase in expression of macrophage markers can be used as a biomarker to identify subgroups of patients who may benefit from adjunctive treatments targeting TAMs and the tumor microenvironment. The success of therapies directed at reversing the suppressive role of immune cells in adult cancers (Robert C, Thomas L, Bondarenko I. Ipilimumab plus dacarbazine for previously untreated metastatic melanoma. N Engl J Med. 2011; 364:2517-26) and the recent development of anti-CSF1R and other antibodies (Hume D A, MacDonald K P A. Therapeutic applications of macrophage colony-stimulating factor-1 (CSF-1) and antagonists of CSF-1 receptor (CSF-1R) signaling. Blood. 2012; 119:1810-20; Horton H M, et al. Fc-engineered anti-CD40 antibody enhances multiple effector functions and exhibits potent in vitro and in vivo antitumor activity against hematologic malignancies. Blood. 2010; 116:3004-12) suggest opportunities for their application in pediatric SHH medulloblastoma.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A process, comprising providing a first composition comprising a plurality of isolated nucleic acids probes;
contacting the first composition to an RNA sample from a mammalian subject desiring a determination of a subgroup of medulloblastoma, to produce one or more cDNA molecules;
providing a second composition comprising one or more isolated nucleic acids probes comprising a sequence capable of hybridizing to one or more nucleic acids selected from the group of genes consisting of βALCAM, βBCAT1, βCBLN3, βCD163, βCD4, βCSF1R, βCXCR4, βDKK2, βEGR1, βEOMES, βFOXG1, βFSTL5, βGABRA5, βGLI1, βHHIP, βIMPG2, βMMD, βMPP3, βMYC, βNPR3, βOTX2, βPDGFRA, βPDLIM3, βPID1, βPPP1R17, βPYGL, βSFRP1, βSLC6A5, βTERC, βTNC, βWIF1 or a combination thereof;
contacting the second composition with the one or more cDNA molecules to amplify the one or more cDNA molecules;
quantifying the expression level of the one or more genes to determine the subgroup of medulloblastoma to which the sample belongs; and
computing the prediction probabilities of the sample belonging to the subgroup of medulloblastoma.

2. The process of claim 1, wherein the subgroup is selected from the group consisting of Group 3, Group 4, SHH and WNT.

3. The process of claim 1, wherein amplifying comprises quantitative PCR.

4. The process of claim 1, wherein quantifying the expression level comprises obtaining a cycle threshold during quantitative PCR.

5. The process of claim 1, wherein computing prediction probabilities comprises:
(i) computing the probabilities as follows:
$${P}_{Group 4} = \frac{{E}_{Group 4}}{S} ;$$
$${P}_{Group 3} = \frac{{E}_{Group 3}}{S} ;$$
$${P}_{SHH} = \frac{{E}_{SHH }}{S} ;$$
$$and$$
$${P}_{WNT} = \frac{{E}_{WNT}}{S} ;$$
wherein,
S=Σₙ₌₁⁴E_{molecular subgroup}_{ₘ}
E_{molecular subgroup}_{ₘ}=e^{LP}^{_{molecular subgroup}}
LP_{molecular subgroup}_{ₘ}=β₀+Σₙ₌₁³¹(βₙ×ΔCT_{Gene}_{_{g}})
ΔCT_{Gene}_{_{g}}=CT_{HKG}−CT_{Gene}_{_{g}}
CT_{HKG}=\sqrt{(CT_{ACTB})²+(CT_{GAPDH})²};
βₙ is the coefficient that corresponds to a given gene; and
(ii) assigning the sample to a class with the highest prediction probability.

6. The process of claim 5, wherein the βₙ coefficient is selected from the group consisting of:

| | Molecular Subgroups | | | |
|---|---|---|---|---|
| Coefficients | Group 3 | Group 4 | SHH | WNT |
| β_{ALCAM} | −1.367776871 | −1.769739151 | −2.189418316 | 1.008437634 |
| β_{BCAT1} | −0.534636855 | 0.213866964 | 0.262004703 | −2.438541412 |
| β_{CBLN3} | −0.584693193 | −0.526325107 | 0.457666963 | −0.701506913 |
| β_{CD163} | −1.599789381 | −2.10319519 | −0.871770084 | −2.441047907 |
| β_{CD4} | −1.631846786 | −2.065302849 | −1.007242441 | −4.022360802 |
| β_{CSF1R} | −2.156824589 | −2.371630192 | −1.46330893 | −4.144571781 |
| β_{CXCR4} | −0.651280224 | −1.001717687 | 0.021241447 | −0.546590805 |
| β_{DKK2} | −0.464394152 | −0.838721573 | −1.601708055 | 2.867124319 |
| β_{EGR1} | −1.638236642 | −2.622097969 | −1.305633783 | −3.253755808 |
| β_{EOMES} | 0.073895209 | 0.379012287 | −0.234139055 | −0.583475113 |
| β_{FOXG1} | −0.359285325 | −0.330290645 | −1.289734721 | −1.304631114 |
| β_{FSTL5} | −0.833438098 | −0.95774281 | −0.573143303 | −3.281084061 |
| β_{GABRA5} | −0.356030613 | −0.835729241 | −0.760884404 | −1.228668332 |
| β_{GLI1} | −1.090841055 | −1.541170001 | −0.214207545 | −1.936838984 |
| β_{HHIP} | −0.968614578 | −1.109485984 | 0.192008331 | −2.467005014 |
| β_{IMPG2} | −0.778781414 | −1.314459562 | −2.196502209 | −0.081688486 |
| β_{MMD} | −0.450012505 | −0.471098512 | −1.69543767 | −0.79158479 |
| β_{MPP3} | −0.288985938 | 0.107019328 | −0.516841948 | −1.593216777 |
| β_{MYC} | −0.592757523 | −1.460371256 | −1.720372677 | 2.127179146 |
| β_{NPR3} | −1.027135134 | −1.473740816 | −1.092957854 | −3.806810617 |
| β_{OTX2} | 0.083120883 | 0.079927221 | −1.125548124 | 1.005084276 |
| β_{PDGFRA} | −1.242420554 | −1.575850725 | −0.653730929 | −1.136945486 |
| β_{PDLIM3} | −1.095413208 | −1.304201484 | 0.336270869 | −3.527924538 |
| β_{PID1} | −1.075829983 | −1.330710649 | −0.498279959 | −0.467160344 |
| β_{PPP1R17} | −0.680468321 | −0.425746292 | 0.314777136 | −1.143164039 |
| β_{PYGL} | −0.960527718 | −1.57236135 | −2.264456034 | 1.749578953 |
| β_{SFRP1} | −0.362708092 | −0.09727975 | 1.041796327 | −1.942569137 |
| β_{SLC6A5} | 0.236007646 | 0.541368663 | −1.134016871 | 1.745964885 |
| β_{TERC} | −0.695041239 | −0.377499193 | −1.81052649 | −0.301935345 |
| β_{TNC} | −0.593996227 | −1.059043169 | −1.41117394 | 3.022968531 |
| β_{WIF1} | −0.186942488 | −0.042140722 | −1.34875536 | 3.460480213 |
| β₀ (constant) | −108.5010223 | −157.2399902 | −142.4472809 | −228.9062195 |

7. The process of claim 1, wherein the RNA sample is obtained from a subject having or suspected of having a medulloblastoma tumor.

8. The process of claim 1, wherein the prediction probability is used to determine a course of therapy.

9. The process of claim 8, wherein the therapy targets tumor-associated macrophages (TAM).

10. The process of claim 9, wherein the therapy comprises inhibition of TAMs.

11. The process of claim 10, wherein therapy comprises inhibition of CSF1R inhibitor.

12. The process of claim 8, wherein subject in SHH subgroup of medulloblastoma are prescribed decreased radiation therapy or no radiation therapy.

13. The process of claim 8, wherein subject in Group 3 or Group 4 subgroup of medulloblastoma are prescribed normal radiation therapy or increased radiation therapy.

14. A computer system comprising one or more processors wherein the computer system executes a software application implementing the process of claim 5.

15. An article comprising one or more non-transitory machine-readable media storing instructions operable to cause one or more machines to perform operations, wherein the operations comprise implementing the process of claim 5.

16. A non-transitory computer readable recording medium including programmed instructions, wherein the instructions, when executed by a computer that includes a display unit for displaying the probability of a subtype/subgroup of medulloblastoma, causes the computer to execute the process of claim 5.

17. Non-transitory computer readable media comprising instructions executable by one or more processors that when executed by one or more processors cause the one or more processors to perform the process of claim 5.
